(19) United States
(12) Patent Application Publication
(10) Pub. No.: US 2025/0003012 A1
(43) Pub. Date: Jan. 2, 2025

(54) METHODS, COMPOSITIONS, AND SYSTEMS TO DETECT HEAD AND NECK CANCER IN SALIVA SAMPLES

(71) Applicant: Laboratory Corporation pf America Holdings, Burlington, NC (US)

(72) Inventor: Gerald J. Wallweber, Foster City, CA (US)

(21) Appl. No.: 18/758,520

(22) Filed: Jun. 28, 2024

Related U.S. Application Data
(60) Provisional application No. 63/511,542, filed on Jun. 30, 2023.

(51) Int. Cl. C12Q 1/6886; A61B 10/00; C12Q 1/6851

(52) U.S. Cl. CPC C12Q 1/6886; A61B 10/0051; C12Q 1/6851; C12Q 2600/158

(57) ABSTRACT

Disclosed are methods, compositions, and systems to detect head and neck cancer using saliva samples. For example, the method may measure the presence and/or amount of a biomarker associated with Head and Neck Squamous Cell Carcinoma (HNSCC) in an individual by obtaining a saliva sample from the individual and measuring in the saliva sample an amount of an expression product from at least one gene encoding the biomarkers associated with HNSCC, wherein the genes comprise at least one of AIM2, CDSN, INHBA, MMP1, MMP9, or MMP10.

### CROSS-REFERENCE TO RELATED APPLICATION

This application claims the benefit of U.S. Provisional Application No. 63/511,542, filed Jun. 30, 2023, the entirety of each of which is hereby incorporated by reference.

### FIELD

Disclosed are methods, compositions, and systems to detect head and neck cancer using saliva samples.

### BACKGROUND

Head and neck cancer is a common disease. The American Cancer Society, relying on information from the Surveillance, Epidemiology, and End Results (SEER) database, maintained by the National Cancer Institute (NCI), determined that early detection of oral cavity and oropharyngeal cancer improves patient survival rates (https://www.cancer.org/cancer/oral-cavity-and-oropharyngeal-cancer/detection-diagnosis-staging/survival-rates.html). Also, the American Dental Association recognizes saliva as a biofluid for diagnostic purposes, including for evaluating the risk of head and neck cancer (https://www.ada.org/resources/research/science-and-research-institute/oral-health-topics/salivary-diagnostics).

The majority of head and neck cancers histologically belong to the squamous cell type and hence are categorized as Head and Neck Squamous Cell Carcinoma (HNSCC). HNSCC is the sixth most common cancer world-wide and the third most common in the developing world. The biological mechanisms behind HNSCC are unknown and there are few, if any, biomarkers that provide a reliable indication of this condition. Still, it would be helpful for individuals having susceptibility to HNSCC to adjust their lifestyle so as to avoid triggering an onset of symptoms and/or promoting further progression of the disease. Thus, there is a need to develop and evaluate improved biomarkers for HNSCC.

### SUMMARY

The terms “invention,” “the invention,” “this invention” and “the present invention,” as well as “disclosure,” “this disclosure,” and “the present disclosure” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims and the specification, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Disclosed are methods, compositions and systems to detect head and neck cancer in a subject using a saliva sample. The disclosed methods, compositions and systems may be embodied in a variety of ways.

For example, in certain embodiments the method may comprise measuring the presence and/or amount of a biomarker associated with Head and Neck Squamous Cell Carcinoma (HNSCC) in an individual comprising the steps of: (a) obtaining a saliva sample from the individual; and (b) measuring in the saliva sample an amount of an expression product from at least one gene encoding a biomarker associated with HNSCC, wherein the gene comprises at least one of AIM2, CDSN, INHBA, MMP1, MMP9, or MMP10. Additionally, the expression product of other genes including at least one of MMP13, CRISP3, MUC21, ADAM12, MMP3 or ISG15 may be measured. In yet other embodiments, disclosed is a composition for detection of a biomarker associated with Head and Neck Squamous Cell Carcinoma (HNSCC) in an individual comprising a reagent for detection of at least one of AIM2, CDSN, INHBA, MMP1, MMP9, or MMP10 in saliva. Additionally, the composition may comprise a reagent for detection of at least one of MMP13, CRISP3, MUC21, ADAM12, MMP3 or ISG15. Also disclosed are systems for performing the methods and/or using the compositions of the invention.

### BRIEF DESCRIPTION OF THE DRAWINGS

The invention may be better understood by reference to the following non-limiting figures.

FIG. 1 shows an overview of an embodiment of a method of the disclosure, including a range of sample volumes received for use with the method in accordance with an embodiment of the disclosure.

FIG. 2 shows the gender, age, cancer stage, location of tumor and number of saliva samples (“Total Number”) in accordance with an embodiment of the disclosure.

FIG. 3 shows example of a random forest analysis to identify and rank differentially expressed genes to differentiate HNC from non-cancer samples using the RNASeq dataset showing the top 10 candidate genes.

FIG. 4 shows example of a random forest analysis to identify and rank additional differentially expressed genes to differentiate HNC from non-cancer samples.

FIG. 5 shows a random forest analysis to identify and rank genes that are differentially expressed in saliva to differentiate HNC from non-cancer samples in accordance with an embodiment of the disclosure.

FIG. 6 shows the level of gene expression in copies/μL (y-axis) for each saliva sample for 26 genes (upper panel) as well as for RPL30 (a housekeeping gene) (lower panel) in accordance with an embodiment of the disclosure. The x-axis identifies the gene measured in the saliva sample (upper panel) and its corresponding housekeeping control. The dotted line across the bottom of both graphs labeled as “No Call” represents a ddPCR result from a saliva sample with too few positive droplets obtain the copies/μL.

FIG. 7 shows median normalized expression from 26 genes in normal (i.e., non-cancer subjects) saliva compared to what was reported in the TCGA HNC RNASeq dataset for normal (i.e., non-cancerous) tissue samples in accordance with an embodiment of the disclosure.

FIG. 8 shows median fold-change in expression as calculated separately for early oral cavity cancer (Early OC, stage I/II) and late oral cavity cancer (Late OC, stage III/IV) for saliva samples (left graph) and the TCGA HNC RNASeq dataset (tissue) (right graph) for each of 26 genes in accordance with an embodiment of the disclosure.

FIG. 9 shows normalized ddPCR and the effect of low levels of a normalization gene (RPL30) in certain samples in accordance with an embodiment of the disclosure.

FIG. 10 shows establishing a cutoff for a normalization (e.g., housekeeping gene) where the left panel shows an increase in the range between the upper and lower 95% confidence interval (CI) value at low values of the housekeeping gene (RPL30) and the right panel shows the percent coefficient of variation (% CV) for the housekeeping gene (RPL30) from samples taken from healthy volunteers, or individuals with early oral cancer (OC), or late OC in accordance with an embodiment of the disclosure.

FIG. 11 shows statistical differences between normalized ddPCR expression levels from the saliva of healthy volunteers (HV) compared to the saliva from early oral cavity (Early OC) cancer patients evaluated using an unpaired t-test and Wilcox Rank Sum Test in accordance with an embodiment of the disclosure.

FIG. 12 shows the performance of gene expression levels to classify a saliva sample from either a healthy volunteer or from an early oral cavity cancer patient evaluated by Receiver Operator Characteristic (ROC) analysis in accordance with an embodiment of the disclosure.

FIG. 13 shows gene expression levels combined by logistic regression and performance evaluated by ROC analysis in accordance with an embodiment of the disclosure.

FIG. 14 shows a system in accordance with an embodiment of the disclosure.

FIG. 15 illustrates a computing system in accordance with an embodiment of the disclosure.

### DETAILED DESCRIPTION

### Terms and Definitions

In order for the disclosure to be more readily understood, certain terms are first defined. Additional definitions for the following terms and other terms are set forth throughout the specification.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “and/or” generally is used to refer to at least one or the other. In some cases the term “and/or” is used interchangeably with the term “or.” The term “including” is used herein to mean, and is used interchangeably with, the phrase “including but not limited to.” The term “such as” is used herein to mean, and is used interchangeably with, the phrase “such as but not limited to.”

As used herein, the term “biomarker” or “marker” refers to one or more nucleic acids (e.g., mRNA, DNA or other nucleic acids), polypeptides and/or other biomolecules (e.g., cholesterol, lipids) that can be used to diagnose, or to aid in the diagnosis or prognosis of a disease or syndrome of interest, either alone or in combination with other biomarkers; monitor the progression of a disease or syndrome of interest; and/or monitor the effectiveness of a treatment for a syndrome or a disease of interest.

As used herein, “digital PCR” or “dPCR” refers to the technique whereby individual PCR reactions are partitioned into several hundred to millions of individual wells or, as in “droplet digital PCR” or “ddPCR,” small volume water-oil emulsion droplets. Following PCR amplification, each partition is counted as either positive or negative. The ratio of positive partitions (k) over the total number of partitions (n) is used to calculated the initial concentration (C) with a Poisson distribution as C=−ln(1−k/n).

As used herein, the term “duplex digital drop PCR” or “duplex ddPCR” refers to the ability of the detection system to detect two different colored dyes simultaneously in one ddPCR reaction. Also, as used herein the term “multiplex digital drop PCR” or multiplex ddPCR refers the ability of the detection system to detect multiple different PCR reactions using multiple different colored dyes simultaneously in one ddPCR reaction.

As used herein “Head and Neck Squamous Cell Carcinoma” or “HNSCC” and “Head and Neck Cancer” or “HNC” are used interchangeably to refer to head and neck cancer. Head and neck cancer is the name for cancers that develop in the mouth, nose and sinuses, salivary glands, throat and larynx. Most head and neck cancers are squamous cell cancers They begin in the moist tissues that line the head and neck. The cancer cells may spread into deeper tissue as the cancer grows. There are other cancers that develop in the head and neck, such as brain cancer, eye cancer and esophageal cancer. These other cancers are usually not considered to be head and neck cancers, because those types of cancer and their treatments are different.

As used herein, “Receiver Operator Characteristic” or “ROC” analysis. The receiver operating characteristic (ROC) curve, which is defined as a plot of test sensitivity as the y coordinate versus specificity or false positive rate (FPR) as the x coordinate.

### Overview

Disclosed are methods, compositions and systems for saliva-based screening test for cancers of the oral cavity and oropharynx. Saliva-based screening test for cancers of the oral cavity and oropharynx may be highly advantageous for early HNSCC detection. Saliva is a convenient biological sample for diagnostic purposes and is a biological source that is in close proximity to tissues that may exhibit HNSCC. In certain embodiments, a simple collection device may be used to collect saliva during an annual physical exam with a primary care physician, a six-month preventive dental exam with a dentist, or for at-home saliva collection. The relative ease and noninvasive sample collection makes saliva an ideal biofluid. For HNSCC detection, saliva-based based detection may improve detection sensitivity due to the direct contact with tissues of the oral cavity and oropharynx.

Accordingly, provided in the present disclosure are methods, compositions, and systems (e.g., kits and/or computer software) for diagnosing the presence or increased risk of developing HNSCC. The methods, compositions and systems of the present disclosure may be used to obtain or provide genetic information from a subject in order to objectively diagnose the presence or increased risk for that subject, or other subjects to develop HNSCC. The methods, compositions, and systems according to the present disclosure may be used to determine the presence or increased risk for a subject to develop HNSCC. The methods, compositions and systems may be embodied in a variety of ways.

### Methods for Diagnosing HNSCC

Embodiments of the present invention comprise methods for diagnosing the presence or increased risk of developing HNSCC. The methods may be embodied in a variety of ways.

For example, disclosed is a method to measure the presence and/or amount of a biomarker associated with Head and Neck Squamous Cell Carcinoma (HNSCC) in an individual comprising the steps of: (a) obtaining a saliva sample from the individual; and (b) measuring in the saliva sample an amount of an expression product from at least one gene encoding a biomarker associated with HNSCC, wherein the genes comprise at least one of AIM2, CDSN, INHBA, MMP1, MMP3, or MMP10. Additionally, the expression product of other genes including at least one of MMP13, CRISP3, MUC21, ADAM12, MMP3 or ISG15 may be measured.

In other embodiments, disclosed is a method of identifying an individual at risk for Head and Neck Squamous Cell Carcinoma (HNSCC) comprising: (a) obtaining a saliva sample from the individual; and (b) measuring in the saliva sample an amount of an expression product from at least one gene encoding the biomarkers associated with HNSCC, wherein the genes comprise at least one of AIM2, CDSN, INHBA, MMP1, MMP3, or MMP10, wherein the presence of an altered level of the expression product from the biomarker associated with HNSCC as compared to a control identifies the individual as being at risk for HNSCC. Additionally, the expression product of other genes including at least one of MMP13, CRISP3, MUC21, ADAM12, MMP3 or ISG15 may be measured and compared to controls.

In yet other embodiments, disclosed is a method of identifying an individual with Head and Neck Squamous Cell Carcinoma (HNSCC) and treating the individual comprising the steps of: (a) obtaining a saliva sample from the individual; (b) measuring in the saliva sample an altered amount of an expression product from at least one gene encoding the biomarkers associated with HNSCC as compared to a control, wherein the genes comprise at least one of AIM2, CDSN, INHBA, MMP1, MMP3, or MMP10; and (c) administering to the individual one or more HNSCC treatments. Additionally, the expression product of other genes including at least one of MMP13, CRISP3, MUC21, ADAM12, MMP3 or ISG15 may be measured and compared to controls for altered expression.

For example, depending on the site and extent of the primary tumor and the status of the lymph nodes, some general considerations for the treatment of lip and oral cavity cancer include the following: surgery alone, radiation therapy alone, a combination of both. See e.g., Hlarrison L B, Sessions R B, Hong W K, eds.: Head and Neck Cancer: A Multidisciplinary Approach. 3rd ed. Lippincott, William & Wilkins, 2009; see also information available from the National Cancer Institute found at https://www.cancer.gov/types/head-and-neck/hp. In certain embodiments, an optimal approach for the treatment of oropharyngeal cancer may not be easily defined because no single regimen offers a clear-cut, superior-survival advantage. Treatment considerations should account for functional and performance status including speech and swallowing outcomes. Treatments include surgery, radiation therapy, chemotherapy, and immunotherapy.

Yet other embodiments of the disclosure include a method of identifying an individual at risk for Head and Neck Squamous Cell Carcinoma (HNSCC) and monitoring the individual, comprising the steps of: (a) obtaining a saliva sample from the individual; (b) measuring in the saliva sample an altered amount of an expression product from at least one gene encoding the biomarkers associated with HNSCC, wherein the genes comprise at least one of AIM2, CDSN, INHBA, MMP1, MMP9, or MMP10; (c) determining that at least one of AIM2, CDSN, INHBA, MMP1, MMP9, or MMP10 have altered expression as compared to a healthy control; and (d) repeating steps (a)-(c) at a later time-point to determine if the at least one of AIM2, CDSN, INHBA, MMP1, MMP9, or MMPP10 shows an increase in altered expression as compared to a healthy control. Additionally, the expression product of other genes including at least one of MMP13, CRISP3, MUC21, ADAM12, MMP3 or ISG15 may be measured in step (b) and evaluated in steps (c)-(d).

In some cases, increasing the number of biomarkers improves the statistical power of the method. In certain embodiments, the methods may comprise measuring the expression product from at least two, or three, or four, or five or all of AIM2, CDSN, INHBA, MMP1, MMP9, or MMP10. Additionally, the expression product of other genes including at least one, or at least two, or at least three, or at least four, or at least five or all of MMP13, CRISP3, MUC21, ADAM12, MMP3 or ISG15 may be measured in combination with each other or with at least one of AIM2, CDSN, INHBA, MMP1, MMP9, or MMP10. In certain embodiments, the methods may comprise measuring expression of CDSN and AIM2, and/or CDSN, AIM2 and MMP1, and/or CDSN, AIM2, MMP1 and INHBA, and/or CDSN, AIM2, MMP1, INHBA and/or MMP9. Or other gene combinations may be measured.

Or other combinations of the disclosed markers may be used. In an embodiment, to determine a preferred combination of biomarkers, one approach is to perform a ROC analysis with each of the markers individually, and as pairs, and as groups of three, groups of four, groups of five and all six (or more) together. For example, using this approach with six genes, there are a total of 63 combinations possible. Thus, six genes (n=6) individually (k=1)=6!/[1!x(6−1)!]=6 combinations; six genes (n=6) as pairs (k=2)=6!/[2!x(6−2)!]=15 combinations; six genes (n=6) in groups of threes (k=3)=6!/[3!x(6−3)!]=20 combinations; six genes (n=6) in groups of four (k=4)=6!/[4!x(6−4)!]=15 combinations; six genes (n=6) in groups of five (k=5)=6!/[5!x(6−5)!]=6 combinations; and six genes (n=6) of all six (k=6)=6!/[6!x(6−6)!]=1 combination such that all together there are 6+15+20+15+6+1=63 combinations. Under a similar analysis, for seven genes there are 127 possible combinations; for eight genes there are 255 possible combinations; for nine genes there are 511 possible combinations; and for ten genes there are 1,023 possible combinations.

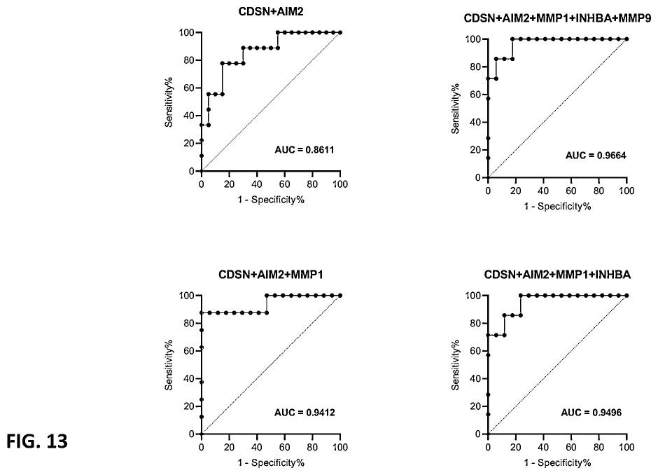

For example, FIG. 13 shows some of the combinations with the AUC for certain of the disclosed markers. A perfect test, one with 100% sensitivity and 100% specificity, would have and AUC=1. It can be seen that combinations with AUCs 0.8611, 0.9664, 0.9412 and 0.9496 for all 4 combinations are within 15% and three of the combinations are within 10% of a perfect test.

Additionally, in certain embodiments, other biomarkers may be measured. Thus, in certain embodiments, the method may further comprise measuring the expression product from any of the genes shown in Tables 1, 2, 5 or 6. Biomarkers in Table 2 (herein) are from Table 6 of commonly owned U.S. application Ser. No. 16/224,974, filed Dec. 19, 2018 and published as US 2019/0187143 A1 (incorporated by reference in its entirety herein).

As disclosed in detail herein, in certain embodiments, the gene expression of the biomarker is normalized. For example, in certain embodiments, the method further comprises measuring the expression product of a housekeeping gene and normalizing the results. The normalizing or housekeeping gene may be RPL30. Or the normalizing or housekeeping gene may be KHDRBS1. Or another housekeeping or normalizing expression product may be used.

In various embodiments, the expression product is a protein or an nucleic acid. In certain embodiments, the expression product is mRNA. In certain embodiments, the measuring comprises measuring the amount mRNA. Or, the measuring may comprise an immunoassay.

A variety of methods may be used to measure the expression product or products. In an embodiment, the method provide quantitative results. In certain embodiments, the method used to measure expression comprises real-time reverse transcriptase PCR (e.g., real-time RT-PCR), droplet digital PCR (ddPCR), duplex droplet digital PCR (duplex-ddPCR) or multiplex droplet digital PCR (multiplex-ddPCR).

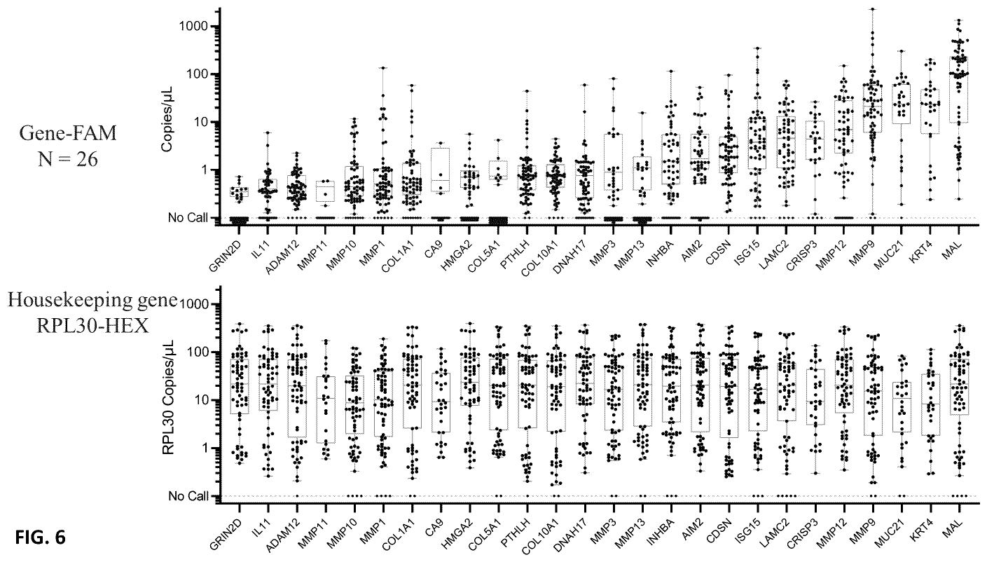

Duplex droplet digital PCR refers to the ability of the detection system to detect two different colored dyes simultaneously in one ddPCR reaction. Thus, in certain embodiments, the method may comprise using a ddPCR probe labeled with a first dye (e.g., HEX) for a housekeeping or control gene (e.g., RPL30) and a second ddPCR probe labeled with a second dye (e.g., FAM) for the gene of interest (e.g. FIG. 6). These two reactions may be performed simultaneously, in the same reaction tube, and detected simultaneously using, for example, a commercial droplet reader such as, but not limited to, a BioRad QX200 ddPCR droplet reader. Additionally and/or alternatively, commercial droplet readers that have the capability to detect more than two dyes simultaneously may be used. For example, the QX600 droplet digital reader from BioRad enables six-color multiplexing.

Additionally and/or alternatively, the method may comprise using an array of expression products. Or other methods including, but not limited to, Northern blot, dot blot, ribonuclease protection assays (RPAs), serial analysis of gene expression (SAGE), differential or subtractive hybridization, reverse transcriptase PCR (RT-PCR), microarrays, next generation sequencing (NGS) and/or RNA-Seq may be used.

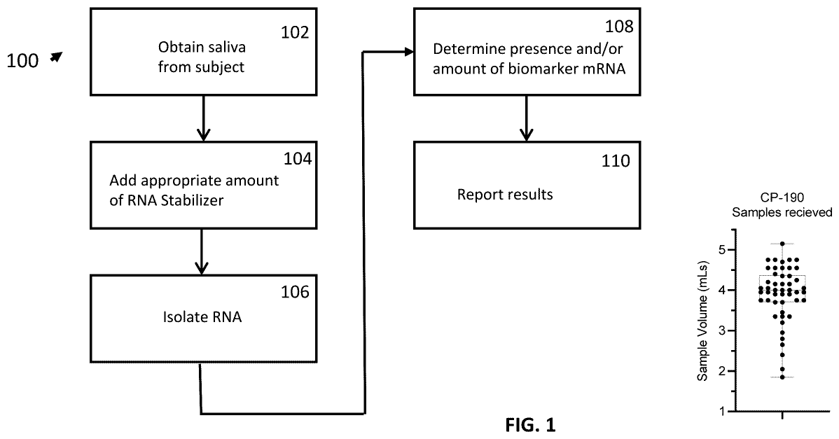

FIG. 1 shows an overview of an embodiment of a method 100 of the disclosure. Thus, as shown in FIG. 1 the method may comprise obtaining a saliva sample from a subject 102. A simple collection device, such as one similar to the DNA Genotek CP-190, may be used to collect saliva during an annual physical exam with a primary care physician, a six-month preventive dental exam with a dentist, or for at home collection. Or other collection devices may be used. The relative ease and noninvasive sample collection makes saliva an ideal biofluid.

The method may further include steps to prepare the sample for analysis of the expression product. In certain embodiments, the measuring comprises measuring mRNA. Where the expression product is mRNA the method may include adding an appropriate amount of an RNA stabilizer 104. For example, in certain embodiments, an equal volume (e.g. 2 mL) of stabilizer is added to a 2 mL aliquot of sample. The samples that include the added stabilizer can be stored at room temperature (RT) for up to 8 weeks, or ≤20° C. long term. Collection devices may be shipped at ambient temperature, processed following manufacturer instructions and stored at ≤70° C. FIG. 1 insert shows the range of sample volumes that may be received in an embodiment of the method.

Next the mRNA may be isolated 106. For example, an aliquot of saliva/stabilization fluid may be removed from the saliva collection device and total RNA isolated, as for example, using a MagMax mirVanna™ Total RNA Isolation kit on a KingFisher™ Flex Purification System. Or other methods of RNA isolation may be used.

Next, the amount of the mRNA may be measured using a quantitative technique 108. For example, in certain embodiments, and as disclosed in detail herein, duplex-ddPCR may be used. Thus, in certain embodiments, an aliquot of the eluent from the RNA isolation procedure may be used to for the synthesis of first-strand cDNA using random hexamers. Next, amplification reaction mixtures may be prepared using target gene primers/probes (in some cases where the probe(s) is labeled with a detectable moiety such as e.g., FAM) and a housekeeping gene (e.g., RPL30) primers/probe (in some cases where the probe is labeled with a different detectable moiety than the gene-specific probe, such as e.g., HEX). Droplets may be made, e.g., using a commercial droplet generator, and plates sealed with a pierceable foil. Next, thermal cycling (i.e., PCR amplification) may be performed. Droplets may then be detected and analyzed using an analysis software that may report units in copies/μL. In various embodiments, control values for the genes of interest may be measured using a sample (or samples) of normal (non-cancerous tissue or saliva or other body fluid) or may be derived from a normal (non-cancerous) population. Additionally and/or alternatively, as noted above, the method may include measurement of at least one normalization (e.g., housekeeping) gene. The housekeeping gene may be measured using the patient sample to allow for normalization of the level of gene expression. In an embodiment, the normalization gene may be KHDRBS1. In other embodiments, RPL30 or other normalization genes may be used.

At this point, the results may be reported 110 to the subject or his or her health care provider.

### Compositions

As noted above, yet other embodiments of the invention comprise compositions to detect biomarkers associated with HNSCC in an individual. The compositions may be embodied in a variety of ways.

Thus, other aspects of the disclosure comprise a composition for detecting or measuring a biomarker associated with Head and Neck Squamous Cell Carcinoma (HNSCC) in an individual comprising a reagent for detection of at least one of AIM2, CDSN, INHBA, MMP1, MMP9, or MMPP10 in saliva. Additionally, the expression product of other genes including at least one of MMP13, CRISP3, MUC21, ADAM12, MMP3 or ISG15 may be measured. Thus, in certain embodiments, the composition comprises a reagent for detection of at least one of MMP13, CRISP3, MUC21, ADAM12, MMP3 or ISG15. Additionally, in certain embodiments, other biomarkers may be measured. Thus in certain embodiments, the composition and/or kit may comprise reagents for measuring the expression product from any of the genes shown in Tables 1, 2, 5 or 6.

In some cases, increasing the number of biomarkers improves the statistical power of the method. In certain embodiments, the compositions may comprise reagents for measuring the expression product from at least two, or three, or four, or five or all of AIM2, CDSN, INHBA, MMP1, MMP9, or MMP10. Additionally and/or alternatively, the composition may comprise a reagent for measuring the expression product of other genes including at least one, or at least two, or at least three, or at least four, or at least five or all of MMP13, CRISP3, MUC21, ADAM12, MMP3 or ISG15 in combination with each other or with at least one of AIM2, CDSN, INHBA, MMP1, MMP9, or MMP10. In certain embodiments, the composition may comprise reagents for measuring expression of CDSN and AIM2, and/or CDSN, AIM2 and MMP1, and/or CDSN, AIM2, MMP1 and INHBA, and/or CDSN, AIM2, MMP1, INHBA and/or MMP9. Or reagents for measuring other gene combinations may be used.

As disclosed in detail herein, in certain embodiments, the gene expression of the biomarker is normalized. For example, in certain embodiments, the composition further comprises reagents for measuring the expression product of a housekeeping gene in saliva and normalizing the results. The normalizing or housekeeping gene may be RPL30. Or the normalizing or housekeeping gene may be KHDRBS1. Or another housekeeping or normalizing expression product may be used.

In various embodiments, the expression product is a protein or an nucleic acid. In certain embodiments, the expression product is mRNA. In certain embodiments, the composition comprises reagents for measuring mRNA. A variety of methods may be used to measure the expression product or products. In certain embodiments, the composition and/or kit may comprise reagents to perform duplex-ddPCR and/or multiplex ddPCR. Additionally and/or alternatively, the composition may comprise an array for measurement of expression products. Or other methods as disclosed herein may be used. Or, the composition and/or kit may comprise reagents for measuring proteins as for example, using an immunoassay.

Thus, the composition may, in certain embodiments, comprise primers (e.g. primer pairs) and/or probes for any one of these genes, where the primers and/or probes are labeled with a detectable moiety as described herein. Additionally and/or alternatively, the primers and/or probes may also comprise an array wherein the primers and/or probes are immobilized on a surface. In other embodiments, the reagents may comprise reagents to measure peptides and/or proteins expressed from the disclosed genes. For example, the composition may comprise reagents to perform an immunoassay. These reagents may, in some embodiments, comprise an array as described in detail herein. As described in detail herein, the reagents may be labeled with a detectable moiety.

In certain embodiments, the composition comprises reagents to quantify the levels of at least one of the disclosed biomarkers in a biological sample. For example, as described in detail herein the composition may comprise reagents to quantitatively measure mRNA. A variety of methods may be used to measure the expression product or products. In certain embodiments, the composition comprises reagents to measure expression using one of real-time reverse transcriptase PCR (e.g., real-time RT-PCR), droplet digital PCR (ddPCR), duplex-ddPCR or multiplex-ddPCR.

Thus, in certain embodiments, the composition comprises reagents to analyze an aliquot of the eluent from the RNA isolation procedure by the synthesis of first-strand cDNA using random hexamers. The composition may further comprise reagents to prepare amplification reaction mixtures using target gene primers/probes and a housekeeping gene (e.g., RPL30) primers/probe. For example, using duplex ddPCR, the primers and/or probe for the gene of interest may be labeled with a first detectable moiety (e.g., FAM), and the primers and/or probe for the housekeeping gene may be labeled with a second detectable moiety (e.g., HEX). Or other detectable moieties such as those described in detail herein may be used. The composition may further comprise reagents to form droplets, and to perform PCR amplification. In various embodiments, the compositions may include reagents (e.g., control nucleic acid template) to measure control values from a sample (or samples) of normal (non-cancerous tissue) or derived from a normal (non-cancerous) population. Additionally and/or alternatively, as noted above, the composition may include reagents for measurement of at least one normalization (e.g., housekeeping) gene. In an embodiment, the normalization gene may be KHDRBS1. In other embodiments, RPL30 or other normalization genes may be used.

Or the composition may comprise reagents to measure a peptide or polypeptide biomarkers. In one embodiment, the composition comprises reagents to perform an immunoassay. In an embodiment, the composition comprises reagents to perform a quantitative immunoassay (e.g., a chemiluminescent immunoassay, ELISA or similar quantitative methods). Or, the composition may comprise reagents to perform flow cytometry. Or, as discussed in detail herein, the composition may comprise reagents to determine the presence of a particular sequence and/or expression level of a nucleic acid. As described in detail herein, the reagents may be labeled with a detectable moiety.

### Systems

In certain embodiments, the invention comprises a system for performing any or all of the steps the methods disclosed herein and/or using the compositions described herein. In certain embodiments, the system may comprise a kit. Or, the system may comprise computerized instructions and/or reagents for performing the methods disclosed herein.

Thus, in certain embodiments, disclosed is a system to measure the presence and/or amount of a biomarker associated with Head and Neck Squamous Cell Carcinoma (HNSCC) in a saliva sample from an individual comprising: (a) a station and/or component for obtaining a saliva sample from the individual; and (b) a station and/or component for measuring in the saliva sample the presence and/or an amount of an expression product from at least one gene encoding the biomarkers associated with HNSCC, wherein the genes comprise at least one of AIM2, CDSN, INHBA, MMP1, MMP9, or MMP10. Additionally, the expression product of other genes including at least one of MMP13, CRISP3, MUC21, ADAM12, MMP3 or ISG15 in saliva may be measured using the system. Thus, in certain embodiments, the system comprises a station and/or component for detection of the presence and/or an amount of at least one of MMP13, CRISP3, MUC21, ADAM12, MMP3 or ISG15. Additionally, in certain embodiments, other biomarkers may be measured. Thus in certain embodiments, the system may comprise a station and/or component for measuring the expression product from any of the genes shown in Tables 1, 2, 5, or 6.

In other embodiments, disclosed is a system to identify an individual at risk for Head and Neck Squamous Cell Carcinoma (HNSCC) comprising: (a) a station and/or component for obtaining a saliva sample from the individual; and (b) a station and/or component for measuring in the saliva sample the presence and/or an amount of an expression product from at least one gene encoding the biomarkers associated with HNSCC, wherein the genes comprise at least one of AIM2, CDSN, INHBA, MMP1, MMP9, or MMP10, wherein the presence of an altered level of the expression product from the biomarker associated with HNSCC as compared to a control identifies the individual as being at risk for HNSCC. Or other gene expression products may be measured. In certain embodiments, the system comprises a station and/or component for detection of the presence and/or amount at least one of MMP13, CRISP3, MUC21, ADAM12, MMP3 or ISG15. Additionally, in certain embodiments, other biomarkers may be measured.

Thus in certain embodiments, the system may comprise a station and/or component for measuring the expression product from any of the genes shown in Tables 1, 2, 5 or 6.

As discussed in detail herein, in certain embodiments, various combinations of the genes may be measured using the disclosed systems. In some cases, increasing the number of biomarkers improves the statistical power of the method. In certain embodiments, the system may comprise a station and/or component for measuring the expression product from at least two, or three, or four, or five or all of AIM2, CDSN, INHBA, MMP1, MMP9, or MMP10. Additionally and/or alternatively, the system may comprise a station and/or component for measuring the expression product of other genes including at least one, or at least two, or at least three, or at least four, or at least five or all of MMP13, CRISP3, MUC21, ADAM12, MMP3 or ISG15 in combination with each other or with at least one of AIM2, CDSN, INHBA, MMP1, MMP9, or MMP10. In certain embodiments, the system may comprise a station and/or component for measuring expression of CDSN and AIM2, and/or CDSN, AIM2 and MMP1, and/or CDSN, AIM2, MMP1 and INHBA, and/or CDSN, AIM2, MMP1, INHBA and/or MMP9. Or stations and/or components for measuring other gene combinations may be used.

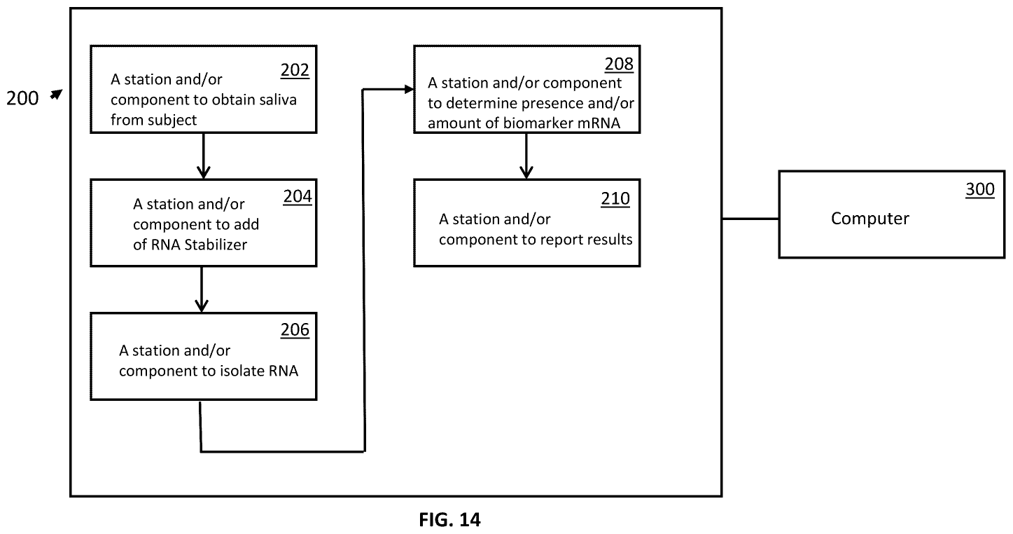

FIG. 14 shows an overview of an embodiment of a system 200 of the disclosure. Thus, as shown in FIG. 14, the system may comprise a station and/or component for obtaining a saliva sample from a subject 202. A simple collection device, such as one similar to the DNA Genotek CP-190, may be used to collect saliva during an annual physical exam with a primary care physician, a six-month preventive dental exam with a dentist, or for at home collection. Or other collection devices may be used. The relative ease and noninvasive sample collection makes saliva an ideal bio-fluid.

The system may further include stations and/or components to prepare the sample for analysis of the expression product. In certain embodiments, the measuring comprises measuring mRNA. Where the expression product is mRNA the system may include a station and/or component for adding an appropriate amount of an RNA stabilizer 204. For example, in certain embodiments, an equal volume (e.g. 2 mL) of stabilizer is added to a 2 mL aliquot of sample. The samples that include the added stabilizer can be stored at room temperature (RT) for up to 8 weeks, or ≤20° C. long term. Collection devices may be shipped at ambient temperature, processed following manufacturer instructions, and stored at ≤70° C.

The system may further comprise a station and/or component for isolation of mRNA 206. For example, an aliquot of saliva/stabilization fluid may be removed from the saliva collection device and total RNA isolated, as for example, using a MagMax mirVanna™ Total RNA Isolation kit on a KingFisher™ Flex Purification System. Or other methods of RNA isolation may be used.

The system may further comprise a station and/or component for measuring an amount of the mRNA using a quantitative technique 208. For example, in certain embodiments, and as disclosed in detail herein duplex-ddPCR and/or multiplex-ddPCR may be used. Thus, in certain embodiments, an aliquot of the eluent from the RNA isolation procedure may be used to for the synthesis of first-strand cDNA using random hexamers. Next, amplification reaction mixtures may be prepared using target gene primers/probes (and in some cases where the probe(s) is labeled with a detectable moiety such as e.g., FAM) and a housekeeping gene (e.g., RPL30) primers/probe (in some cases where the probe is labeled with a different detectable moiety than the gene-specific probe, such as e.g., HEX). Droplets may be made, e.g., using a commercial droplet generator, and plates sealed with a pierceable foil. Next, thermal cycling (i.e., PCR amplification) may be performed. Droplets may then be detected and analyzed using an analysis software that may report units in copies/μL. In various embodiments, measuring control values may be from a sample (or samples) of normal (non-cancerous tissue) or derived from a normal (non-cancerous) population. Additionally and/or alternatively, as noted above, the system may include a station and/or component for measurement of at least one normalization (e.g., housekeeping) gene. In an embodiment, the normalization gene may be KHDRBS1. In other embodiments, RPL30 or other normalization genes may be used.

The system may further comprise a station and/or component for reporting the results 210 to the subject or his or her health care provider.

In certain embodiments, the system may comprise a computer 300. Thus, disclosed herein is a computer (e.g., data processor) and/or a computer-program product tangibly embodied in a non-transitory machine-readable storage medium, including instructions configured to run any of the stations/components of the system and/or perform a step or steps of the methods of any of the disclosed embodiments. In one embodiment, the system comprises a computer and/or a computer-program product tangibly embodied in a non-transitory machine-readable storage medium, including instructions configured to measure the presence and/or amount of a biomarker associated with Head and Neck Squamous Cell Carcinoma (HNSCC) in an individual comprising the steps of: (a) obtaining a saliva sample from the individual; and (b) measuring in the saliva sample the presence and/or an amount of an expression product from at least one gene encoding the biomarkers associated with HNSCC, wherein the genes comprise at least one of AIM2, CDSN, INHBA, MMP1, MMP9, or MMP10. Or, as discussed above, other gene expression products may be measured. Thus, in certain embodiments, the computer and/or a computer-program product tangibly embodied in a non-transitory machine-readable storage medium, includes instructions configured to measure the presence and/or amount of at least one of MMP13, CRISP3, MUC21, ADAM12, MMP3 or ISG15. Additionally, in certain embodiments, other biomarkers may be measured. Thus in certain embodiments, the computer and/or a computer-program product tangibly embodied in a non-transitory machine-readable storage medium, includes instructions configured to measure the presence and/or amount of an expression product from any of the genes shown in Tables 1, 2, 5 or 6.

In other embodiments, the system comprises a computer and/or a computer-program product tangibly embodied in a non-transitory machine-readable storage medium, including instructions configured to identify an individual at risk for Head and Neck Squamous Cell Carcinoma (HNSCC) comprising: (a) obtaining a saliva sample from the individual; and (b) measuring in the saliva sample an amount of an expression product from at least one gene encoding the biomarkers associated with HNSCC, wherein the genes comprise at least one of AIM2, CDSN, INHBA, MMP1, MMP9, or MMP10, wherein the presence of an altered level of the expression product from the biomarker associated with HNSCC as compared to a control identifies the individual as being at risk for HNSCC. Or other gene expression products may be measured. Thus, in certain embodiments, the computer and/or a computer-program product tangibly embodied in a non-transitory machine-readable storage medium, includes instructions configured to measure the presence and/or amount of at least one of MMP13, CRISP3, MUC21, ADAM12, MMP3 or ISG15. Additionally, in certain embodiments, other biomarkers may be measured. Thus, in certain embodiments, the computer and/or a computer-program product tangibly embodied in a non-transitory machine-readable storage medium, includes instructions configured to measure the presence and/or amount of an expression product from any of the genes shown in Tables 1, 2, 5 or 6.

In some cases, increasing the number of biomarkers improves the statistical power of the method. In certain embodiments, the computer and/or a computer-program product tangibly embodied in a non-transitory machine-readable storage medium, includes instructions configured to measure the expression product from at least two, or three, or four, or five or all of AIM2, CDSN, INHBA, MMP1, MMP9, or MMP10. Additionally and/or alternatively, the computer and/or a computer-program product tangibly embodied in a non-transitory machine-readable storage medium, includes instructions configured to measure the expression product of other genes including at least one, or at least two, or at least three, or at least four, or at least five or all of MMP13, CRISP3, MUC21, ADAM12, MMP3 or ISG15 in combination with each other or with at least one of AIM2, CDSN, INHBA, MMP1, MMP9, or MMP10. In certain embodiments, the computer and/or a computer-program product tangibly embodied in a non-transitory machine-readable storage medium, includes instructions configured to measure expression of CDSN and AIM2, and/or CDSN, AIM2 and MMP1, and/or CDSN, AIM2, MMP1 and INHBA, and/or CDSN, AIM2, MMP1, INHBA and/or MMP9. Additionally, in certain embodiments, other biomarkers may be measured.

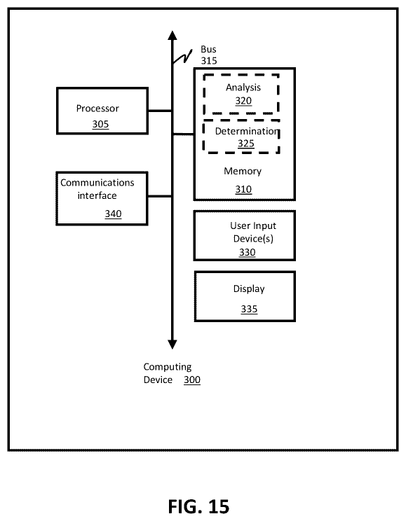

FIG. 15 shows a block diagram of an analysis system 300 used for detection and/or quantification of an analyte from a dried sample. As illustrated in FIG. 15, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 15, modules, engines, or components are shown for implementing the methods or running any of the systems of the disclosure.

Thus, FIG. 15 illustrates an example computing device 300 suitable for use with systems and the methods according to this disclosure. The example computing device 300 includes a processor 305 which is in communication with the memory 310 and other components of the computing device 300 using one or more communications buses 315. The processor 305 is configured to execute processor-executable instructions stored in the memory 310 to perform one or more methods or operate one or more stations for detecting antibodies to SARS-CoV-2 according to different examples, such as those in FIGS. 1-14 or disclosed elsewhere herein. In this example, the memory 310 may store processor-executable instructions 325 that can analyze 320 results for sample as discussed herein.

The computing device 300 in this example may also include one or more user input devices 330, such as a keyboard, mouse, touchscreen, microphone, etc., to accept user input. The computing device 300 may also include a display 335 to provide visual output to a user such as a user interface. The computing device 300 may also include a communications interface 340. In some examples, the communications interface 340 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

As disclosed in detail herein, in certain embodiments, the gene expression of the biomarker is normalized. For example, in certain embodiments, the system further comprises measuring the expression product of a housekeeping gene and normalizing the results. The normalizing or housekeeping gene may be RPL30. Or the normalizing or housekeeping gene may be KHDRBS1. Or another housekeeping or normalizing expression product may be used.

In various embodiments, the expression product is a protein or an nucleic acid. In certain embodiments, the expression product is mRNA. In certain embodiments, the measuring comprises measuring the amount mRNA. Or, the measuring may comprise an immunoassay.

A variety of methods may be used by the system to measure the expression product or products. In an embodiment, the method provide quantitative results. In certain embodiments, the method used to measure expression comprises real-time reverse transcriptase PCR (e.g., real-time RT-PCR), droplet digital PCR (ddPCR) or duplex-ddPCR. Additionally and/or alternatively, the method may comprise using an array of expression products. Or other methods as disclosed

In certain embodiments, the disclosure provides kits for use in accordance with methods and compositions disclosed herein. Generally, kits comprise one or more reagents detect the biomarker of interest and optionally, instructions for use. Suitable reagents may include nucleic acid probes and/or antibodies or fragments thereof. In some embodiments, suitable reagents are provided in a form of an array such as a microarray or a mutation panel. Kits may further comprise reagents that serve as positive controls for the biomarkers (i.e., genes) of interest.

Thus, embodiments of the disclosure comprise a kit to detect biomarkers associated with HNSCC in an individual. In certain embodiments, the kit comprises reagents that quantify the levels of at least one of the disclosed biomarkers in a biological sample. For example, as described in detail herein the kit may comprise reagents to measure mRNA. Or the kit may comprise reagents to measure a peptide or polypeptide biomarkers. In one embodiment, the kit comprises reagents to perform an immunoassay. Or the kit may comprise reagents to perform flow cytometry. Or as discussed in detail herein, the kit may comprise reagents to determine the presence of a particular sequence and/or expression level of a nucleic acid. As described in detail herein, the reagents may be labeled with a detectable moiety.

Thus, other aspects of the disclosure comprise a kit for detecting or measuring a biomarker associated with Head and Neck Squamous Cell Carcinoma (HNSCC) in an individual comprising a reagent for detection of at least one of AIM2, CDSN, INHBA, MMP1, MMP9, or MMP10 in saliva. Or other gene expression products may be measured. Thus, in certain embodiments, the kit includes a reagent to measure the presence and/or amount of at least one of MMP13, CRISP3, MUC21, ADAM12, MMP3 or ISG15. Additionally, in certain embodiments, other biomarkers may be measured. Thus, in certain embodiments, the kit comprises a reagent to measure the presence and/or amount of an expression product from any of the genes shown in Tables 1, 2, 5 or 6.

In some cases, increasing the number of biomarkers improves the statistical power. In certain embodiments, the kit may comprise reagents for measuring the expression product from at least two, or three, or four, or five or all of AIM2, CDSN, INHBA, MMP1, MMP9, or MMP10. Or other gene expression products may be measured. Thus, in certain embodiments, the kit includes a reagent to measure the presence and/or amount of at least two, three, four, five or all six of MMP13, CRISP3, MUC21, ADAM12, MMP3 or ISG15 in combination with each other or at least one of AIM2, CDSN, INHBA, MMP1, MMP9, or MMP10. Additionally, in certain embodiments, other biomarkers may be measured. Thus in certain embodiments, the kit comprises a reagent to measure the presence and/or amount of an expression product from at least two, or three or four or more of any the genes shown in Tables 1, 2, 5 or 6. In certain embodiments, the kit may comprise reagents for measuring expression of CDSN and AIM2, and/or CDSN, AIM2 and MMP1, and/or CDSN, AIM2, MMP1 and INHBA, and/or CDSN, AIM2, MMP1, INHBA and/or MMP9.

In various embodiments, the expression product is a protein or an nucleic acid. In certain embodiments, the expression product is mRNA. In certain embodiments, the kit comprises reagents for measuring mRNA. A variety of methods may be used to measure the expression product or products. In certain embodiments, the kit may comprise reagents to perform duplex-ddPCR or multiplex-ddPCR. Additionally and/or alternatively, the kit may comprise an array of expression products. Or other methods as disclosed herein may be used. Or the kit may comprise reagents for measuring proteins as for example, using an immunoassay.

Additionally and/or alternatively, the kit may include a reagent to detect at least one normalization (e.g., housekeeping) gene. In an embodiment, the normalization gene may be KHDRBS1. In other embodiments, RPL30 or other normalization genes may be used. The kit may, in some embodiments, include positive controls for any of the disclosed biomarkers and/or normalization genes as well as controls from normal (i.e., non-cancerous) samples.

Thus, the kit may, in certain embodiments, comprise primers (e.g. primer pairs) and/or probes for any one of these genes, where the primers and/or probes are labeled with a detectable moiety as described herein. Additionally and/or alternatively, the primers and/or probes may also comprise an array wherein the primers and/or probes are immobilized on a surface. In other embodiments, the reagents may comprise reagents to measure peptides and/or proteins expressed from the disclosed genes. For example, the kit may comprise reagents to perform an immunoassay. These reagents may, in some embodiments, comprise an array as described in detail herein. As described in detail herein, the reagents may be labeled with a detectable moiety.

The kit may further comprise instructions for use.

In some embodiments, the provided kits further comprise reagents for carrying out various detection methods described herein (e.g., RT-PCR, sequencing, hybridization, primer extension, multiplex ASPE, immunoassays, etc.). For example, kits may optionally contain buffers, enzymes, and/or reagents for use in methods described herein, e.g., for amplifying nucleic acids via duplex or multiplex ddPCR, RT-PCR (i.e., real-time RT-PCR), primer-directed amplification, for performing ELISA experiments, etc. The kit may, in certain embodiments, comprise primers and/or probes for any one of these genes, where the primers and/or probes are labeled with a detectable moiety as described herein.

In some embodiments, the provided kits further comprise a control indicative of a healthy individual, e.g., a nucleic acid and/or protein sample from an individual who does not have the disease and/or syndrome of interest. Or the kit may comprise a positive control comprising a known amount of one (or more) of the biomarker genes being measured. Kits may also contain instructions on how to determine if an individual has the disease and/or syndrome of interest, or is at risk of developing the disease and/or syndrome of interest.

In some embodiments, provided is a computer readable medium encoding information corresponding to the biomarker of interest. Such computer readable medium may be included in a kit of the invention.

### Peptide, Polypeptide and Protein Assays

In certain embodiments, the biomarker of interest is detected at the protein level (or peptide or polypeptide level), that is, a gene product is analyzed. For example, a protein or fragment thereof can be analyzed by amino acid sequencing methods, or immunoassays using one or more antibodies that specifically recognize one or more epitopes present on the biomarker of interest, or in some cases specific to a mutation of interest. Proteins can also be analyzed by protease digestion (e.g., trypsin digestion) and, in some embodiments, the digested protein products can be further analyzed by 2D-gel electrophoresis.

### Antibody-Based Detection Methods

Specific antibodies that recognize the biomarker of interest can be employed in any of a variety of methods known in the art. Antibodies against particular epitopes, polypeptides, and/or proteins can be generated using any of a variety of known methods in the art. For example, the epitope, polypeptide, or protein against which an antibody is desired can be produced and injected into an animal, typically a mammal (such as a donkey, mouse, rabbit, horse, chicken, etc.), and antibodies produced by the animal can be collected from the animal. Monoclonal antibodies can also be produced by generating hybridomas that express an antibody of interest with an immortal cell line.

In some embodiments, antibodies are labeled with a detectable moiety as described herein.

Antibody detection methods are well known in the art including, but are not limited to, enzyme-linked immunoadsorbent assays (ELISAs) and Western blots. Some such methods are amenable to being performed in an array format.

For example, in some embodiments, the biomarker of interest is detected using a first antibody (or antibody fragment) that specifically recognizes the biomarker. The antibody may be labeled with a detectable moiety (e.g., a chemiluminescent molecule), an enzyme, or a second binding agent (e.g., streptavidin). Or, the first antibody may be detected using a second antibody, as is known in the art.

In certain embodiments, the method may further comprise adding a capture support, the capture support comprising at least one capture support binding agent that recognizes and binds to the biomarker so as to immobilize the biomarker on the capture support. The method may, in certain embodiments, further comprise adding a second binding agent that can specifically recognize and bind to at least some of the plurality binding agent molecules and/or the biomarker on the capture support. In an embodiment, the binding agent that can specifically recognize and bind to at least some of the plurality binding agent molecules and/or the biomarker on the capture support is a soluble binding agent (e.g., a secondary antibody). The second binding agent may be labeled (e.g., with an enzyme) such that binding of the biomarker of interest is measured by adding a substrate for the enzyme and quantifying the amount of product formed.

In an embodiment, the capture solid support may be an assay well (i.e., such as a microtiter plate). Or, the capture solid support may be a location on an array, or a mobile support, such as a bead. Or the capture support may be a filter.

In some cases, the biomarker may be allowed to complex with a first binding agent (e.g., primary antibody specific for the biomarker and labeled with detectable moiety) and a second binding agent (e.g., a secondary antibody that recognizes the primary antibody or a second primary antibody), where the second binding agent is complexed to a third binding agent (e.g., biotin) that can then interact with a capture support (e.g., magnetic bead) having a reagent (e.g., streptavidin) that recognizes the third binding agent linked to the capture support. The complex (labeled primary antibody: biomarker: second primary antibody-biotin: streptavidin-bead may then be captured using a magnet (e.g., a magnetic probe) to measure the amount of the complex.

A variety of binding agents may be used in the methods of the disclosure. For example, the binding agent attached to the capture support, or the second antibody, may be either an antibody or an antibody fragment that recognizes the biomarker. Or, the binding agent may comprise a protein that binds a non-protein target (i.e., such as a protein that specifically binds to a small molecule biomarker, or a receptor that binds to a protein).

In certain embodiments, the solid supports may be treated with a passivating agent. For example, in certain embodiments the biomarker of interest may be captured on a passivated surface (i.e., a surface that has been treated to reduce non-specific binding). One such passivating agent is BSA. Additionally and/or alternatively, where the binding agent used is an antibody, the solid supports may be coated with protein A, protein G, protein A/G, protein L, or another agent that binds with high affinity to the binding agent (e.g., antibody). These proteins bind the Fc domain of antibodies and thus can orient the binding of antibodies that recognize the protein or proteins of interest.

### Nucleic Acid Assays

In certain embodiments, the biomarkers disclosed herein are detected at the nucleic acid level. In one embodiment, the disclosure comprises methods for diagnosing the presence or an increased risk of developing the syndrome or disease of interest (e.g., HNSCC) in a subject.

The method may comprise the steps of obtaining a nucleic acid from a tissue or body fluid sample from a subject and conducting an assay to identify whether there is over-expression of a gene of interest. For example, over-expression of certain gene products may be quantified using reverse transcriptase PCR (RT-PCR). Or, droplet digital PCR (ddPCR), duplex ddPCR or multiplex ddPCR may be used.

Or the method may comprise the steps of obtaining a nucleic acid from a tissue or body fluid sample from a subject and conducting an assay to identify whether there is a variant sequence (i.e., a mutation) in the subject's nucleic acid. In certain embodiments, the method may comprise comparing the variant to known variants associated with the syndrome or disease of interest and determining whether the variant is a variant that has been previously identified as being associated with the syndrome or disease of interest. Or the method may comprise identifying the variant as a new, previously uncharacterized variant. If the variant is a new variant, the method may further comprise performing an analysis to determine whether the mutation is expected to be deleterious to expression of the gene and/or the function of the protein encoded by the gene. The method may further comprise using the variant profile (i.e., the compilation of mutations identified in the subject) to diagnose the presence of the syndrome or disease of interest or an increased risk of developing the syndrome or disease of interest.

Nucleic acid analyses can be performed on genomic DNA, messenger RNA, and/or cDNA. Also, in various embodiments, the nucleic acid comprises a gene, an RNA, an exon, an intron, a gene regulatory element, an expressed RNA, an siRNA, or an epigenetic element. Also, regulatory elements, including splice sites, transcription factor binding, A-I editing sites, microRNA binding sites, and functional RNA structure sites may be evaluated for mutations (i.e., variants). Thus, for each of the methods and compositions of the disclosure, the variant may comprise a nucleic acid sequence that encompasses at least one of the following: (1) A-to-I editing sites; (2) splice sites; (3) conserved functional RNA structures; (4) validated transcription factor binding sites (TFBS); (5) microRNA (miRNA) binding sites; (6) polyadenylation sites; (7) known regulatory elements; (8) miRNA genes; (9) small nucleolar RNA genes encoded in the ROIs; and/or (10) ultra-conserved elements across placental mammals.

In many embodiments, nucleic acids are extracted from a biological sample. In some embodiments, nucleic acids are analyzed without having been amplified. In some embodiments, nucleic acids are amplified using techniques known in the art (such as generating cDNA that is amplified using the polymerase chain reaction (PCR)) and amplified nucleic acids are used in subsequent analyses. Multiplex PCR, in which several amplicons (e.g., from different genomic regions) are amplified at once using multiple sets of primer pairs, may be employed. For example, nucleic acid can be analyzed by sequencing, hybridization, PCR amplification, restriction enzyme digestion, primer extension such as single-base primer extension or multiplex allele-specific primer extension (ASPE), or DNA sequencing. In some embodiments, nucleic acids are amplified in a manner such that the amplification product for a wild-type allele differs in size from that of a mutant allele. Thus, presence or absence of a particular mutant allele can be determined by detecting size differences in the amplification products, e.g., on an electrophoretic gel. For example, deletions or insertions of gene regions may be particularly amenable to using size-based approaches.

Certain exemplary nucleic acid analysis methods are described in detail below.

Analysis of mRNA

In certain embodiments, mRNA is analyzed using droplet-digital PCR, e.g., duplex ddPCR or multiplex ddPCR. In digital PCR, individual PCR reactions are partitioned into several hundred to millions of individual wells or, as in droplet digital PCR (ddPCR), small volume water-oil emulsion droplets. Following PCR amplification, each partition is counted as either positive or negative. The ratio of positive partitions (k) over the total number of partitions (n) is used to calculated the initial concentration (C) with a Poisson distribution as C=−ln(1−k/n).

In certain embodiments, mRNA is analyzed using real-time and/or reverse-transcriptase PCR using methods known in the art and/or commercial reagents and/or kits. “Real-time PCR” or rPCR is a method for detecting and measuring products generated during each cycle of a PCR, which are proportionate to the amount of template nucleic acid prior to the start of PCR. The information obtained, such as an amplification curve, can be used to determine the presence of a target nucleic acid and/or quantitate the initial amounts of a target nucleic acid sequence. The term “real-time PCR” is used to denote a subset of PCR techniques that allow for detection of PCR product throughout the PCR reaction, or in real-time. In some embodiments, rPCR is real time reverse transcriptase (RT) real-time PCR (rRT-PCR).

Reverse transcriptase PCR is used when the starting material is RNA and/or mRNA. RNA is first transcribed into complementary DNA (cDNA) by reverse transcriptase. In rRT-PCR, the cDNA is then used as the template for the qPCR reaction. rRT-PCR can be performed in a one-step method, which combines reverse transcription and PCR in a single tube and buffer, using a reverse transcriptase along with a DNA polymerase. In one-step rRT-PCR, both RNA and DNA targets are amplified using sequence-specific targets. The term “quantitative PCR” encompasses all PCR-based techniques that allow for quantitative or semi-quantitative determination of the initially present target nucleic acid sequences.

The principles of real-time PCR (rPCR) are generally described, for example, in Held et al. “Real Time Quantitative PCR” Genome Research 6:986-994 (1996). Generally, rPCR measures a signal at each amplification cycle. Some rPCR techniques rely on fluorophores that emit a signal at the completion of every multiplication cycle. Examples of such fluorophores are fluorescence dyes that emit fluorescence at a defined wavelength upon binding to double-stranded DNA, such as SYBR green. An increase in double-stranded DNA during each amplification cycle thus leads to an increase in fluorescence intensity due to accumulation of PCR product. Another example of fluorophores used for detection in rPCR are sequence-specific fluorescent reporter probes. The examples of such probes are TAQMAN® probes. The use of sequence-specific reporter probe provides for detection of a target sequence with high specificity, and enables quantification even in the presence of non-specific DNA amplification. Fluorescent probes can also be used in multiplex assays—for detection of several genes in the same reaction—based on specific probes with different-colored labels. For example, a multiplex assay can use several sequence-specific probes, labeled with a variety of fluorophores, including, but not limited to, FAM, JA270, CY5.5, and/or HEX, in the same PCR reaction mixture.

rPCR relies on detection of a measurable parameter, such as fluorescence, during the course of the PCR reaction. The amount of the measurable parameter is proportional to the amount of the PCR product, which allows one to observe the increase of the PCR product “in real time.” Some rPCR methods allow for quantification of the input DNA template based on the observable progress of the PCR reaction. A “growth curve” or “amplification curve” in the context of a nucleic acid amplification assay is a graph of a function, where an independent variable is the number of amplification cycles and a dependent variable is an amplification-dependent measurable parameter measured at each cycle of amplification, such as fluorescence emitted by a fluorophore. As discussed above, the amount of amplified target nucleic acid can be detected using a fluorophore-labeled probe. Typically, the amplification-dependent measurable parameter is the amount of fluorescence emitted by the probe upon hybridization, or upon the hydrolysis of the probe by the nuclease activity of the nucleic acid polymerase. The increase in fluorescence emission is measured in real time and is directly related to the increase in target nucleic acid amplification. In some examples, the change in fluorescence (dRₙ) is calculated using the equation dRₙ=Rₙ₊−Rₙ₋, with Rₙ₊ being the fluorescence emission of the product at each time point and Rₙ₋ being the fluorescence emission of the baseline. The dRₙ values are plotted against cycle number, resulting in amplification plots. In a typical polymerase chain reaction, a growth curve contains a segment of exponential growth followed by a plateau, resulting in a sigmoidal-shaped amplification plot when using a linear scale. A growth curve is characterized by a “cross point” value or “Cₚ” value, which can be also termed “threshold value” or “cycle threshold” (C), which is a number of cycles where a predetermined magnitude of the measurable parameter is achieved. For example, when a fluorophore-labeled probe is employed, the threshold value (Ct) is the PCR cycle number at which the fluorescence emission (dRₙ) exceeds a chosen threshold, which is typically 10 times the standard deviation of the baseline (this threshold level can, however, be changed if desired). A lower Ct value represents more rapid completion of amplification, while the higher Ct value represents slower completion of amplification. Where efficiency of amplification is similar, the lower Ct value is reflective of a higher starting amount of the target nucleic acid, while the higher Ct value is reflective of a lower starting amount of the target nucleic acid. Where a control nucleic acid of known concentration is used to generate a “standard curve,” or a set of “control” Ct values at various known concentrations of a control nucleic acid, it becomes possible to determine the absolute amount of the target nucleic acid in the sample by comparing Ct values of the target and control nucleic acids.

### Allele-Specific Amplification

In some embodiments, for example, where the biomarker for the disease and/or syndrome of interest is a mutation, a biomarker is detected using an allele-specific amplification assay. This approach is variously referred to as PCR amplification of specific allele (PASA) (Sarkar, et al., 1990 Anal. Biochem. 186:64-68), allele-specific amplification (ASA) (Okayama, et al., 1989 J. Lab. Clin. Med. 114:105-113), allele-specific PCR (ASPCR) (Wu, et al. 1989 Proc. Natl. Acad. Sci. USA. 86:2757-2760), and amplification-refractory mutation system (ARMS) (Newton, et al., 1989 Nucleic Acids Res. 17:2503-2516). This method is applicable for single base substitutions as well as micro deletions/insertions.

For example, for PCR-based amplification methods, amplification primers may be designed such that they can distinguish between different alleles (e.g., between a wild-type allele and a mutant allele). Thus, the presence or absence of amplification product can be used to determine whether a gene mutation is present in a given nucleic acid sample. In some embodiments, allele specific primers can be designed such that the presence of amplification product is indicative of the gene mutation. In some embodiments, allele specific primers can be designed such that the absence of amplification product is indicative of the gene mutation.

In some embodiments, two complementary reactions are used. One reaction employs a primer specific for the wild type allele (“wild-type-specific reaction”) and the other reaction employs a primer for the mutant allele (“mutant-specific reaction”). The two reactions may employ a common second primer. PCR primers specific for a particular allele (e.g., the wild-type allele or mutant allele) generally perfectly match one allelic variant of the target, but are mismatched to other allelic variant (e.g., the mutant allele or wild-type allele). The mismatch may be located at/near the 3′ end of the primer, leading to preferential amplification of the perfectly matched allele. Whether an amplification product can be detected from one or in both reactions indicates the absence or presence of the mutant allele. Detection of an amplification product only from the wild-type-specific reaction indicates presence of the wild-type allele only (e.g., homozygosity of the wild-type allele). Detection of an amplification product in the mutant-specific reaction only indicates presence of the mutant allele only (e.g. homozygosity of the mutant allele). Detection of amplification products from both reactions indicate (e.g., a heterozygote). As used herein, this approach will be referred to as “allele specific amplification (ASA).”

Allele-specific amplification can also be used to detect duplications, insertions, or inversions by using a primer that hybridizes partially across the junction. The extent of junction overlap can be varied to allow specific amplification.

Amplification products can be examined by methods known in the art, including by visualizing (e.g., with one or more dyes) bands of nucleic acids that have been migrated (e.g., by electrophoresis) through a gel to separate nucleic acids by size.

### Allele-Specific Primer Extension

In some embodiments, an allele-specific primer extension (ASPE) approach is used to detect a gene mutations. ASPE employs allele-specific primers that can distinguish between alleles (e.g., between a mutant allele and a wild-type allele) in an extension reaction such that an extension product is obtained only in the presence of a particular allele (e.g., mutant allele or wild-type allele). Extension products may be detectable or made detectable, e.g., by employing a labeled deoxynucleotide in the extension reaction. Any of a variety of labels are compatible for use in these methods, including, but not limited to, radioactive labels, fluorescent labels, chemiluminescent labels, enzymatic labels, etc. In some embodiments, a nucleotide is labeled with an entity that can then be bound (directly or indirectly) by a detectable label, e.g., a biotin molecule that can be bound by streptavidin-conjugated fluorescent dyes. In some embodiments, reactions are done in multiplex, e.g., using many allele-specific primers in the same extension reaction.

In some embodiments, extension products are hybridized to a solid or semi-solid support, such as beads, matrix, gel, among others. For example, the extension products may be tagged with a particular nucleic acid sequence (e.g., included as part of the allele-specific primer) and the solid support may be attached to an “anti-tag” (e.g., a nucleic acid sequence complementary to the tag in the extension product). Extension products can be captured and detected on the solid support. For example, beads may be sorted and detected.

### Single Nucleotide Primer Extension

In some embodiments, a single nucleotide primer extension (SNuPE) assay is used, in which the primer is designed to be extended by only one nucleotide. In such methods, the identity of the nucleotide just downstream of the 3′ end of the primer is known and differs in the mutant allele as compared to the wild-type allele. SNuPE can be performed using an extension reaction in which the only one particular kind of deoxynucleotide is labeled (e.g., labeled dATP, labeled dCTP, labeled dGTP, or labeled dTTP). Thus, the presence of a detectable extension product can be used as an indication of the identity of the nucleotide at the position of interest (e.g., the position just downstream of the 3′ end of the primer), and thus as an indication of the presence or absence of a mutation at that position. SNuPE can be performed as described in U.S. Pat. Nos. 5,888,819; 5,846,710; 6,280,947; 6,482,595; 6,503,718; 6,919,174; Piggee, C. et al. Journal of Chromatography A 781 (1997), p. 367-375 (“Capillary Electrophoresis for the Detection of Known Point Mutations by Single-Nucleotide Primer Extension and Laser-Induced Fluorescence Detection”); Hoogendoorn, B. et al., Human Genetics (1999) 104:89-93, (“Genotyping Single Nucleotide Polymorphism by Primer Extension and High Performance Liquid Chromatography”).

In some embodiments, primer extension can be combined with mass spectrometry for accurate and fast detection of the presence or absence of a mutation. See, U.S. Pat. No. 5,885,775 to Haff et al. (analysis of single nucleotide polymorphism analysis by mass spectrometry); U.S. Pat. No. 7,501,251 to Koster (DNA diagnosis based on mass spectrometry). Suitable mass spectrometric format includes, but is not limited to, Matrix-Assisted Laser Desorption/Ionization, Time-of-Flight (MALDI-TOF), Electrospray (ES), IR-MALDI, Ion Cyclotron Resonance (ICR), Fourier Transform, and combinations thereof.

### Oligonucleotide Ligation Assay

In some embodiments, an oligonucleotide ligation assay (“OLA” or “OL”) is used. OLA employs two oligonucleotides that are designed to be capable of hybridizing to abutting sequences of a single strand of a target molecules. Typically, one of the oligonucleotides is biotinylated, and the other is detectably labeled, e.g., with a streptavidin-conjugated fluorescent moiety. If the precise complementary sequence is found in a target molecule, the oligonucleotides will hybridize such that their termini abut, and create a ligation substrate that can be captured and detected. See e.g., Nickerson et al. (1990) Proc. Natl. Acad. Sci. U.S.A. 87:8923-8927, Landegren, U. et al. (1988) Science 241:1077-1080, and U.S. Pat. No. 4,998,617.

### Hybridization Approach

In some embodiments, nucleic acids are analyzed by hybridization using one or more oligonucleotide probes specific for the biomarker of interest and under conditions sufficiently stringent to disallow a single nucleotide mismatch. In certain embodiments, suitable nucleic acid probes can distinguish between a normal gene and a mutant gene. Thus, for example, one of ordinary skill in the art could use probes of the invention to determine whether an individual is homozygous or heterozygous for a particular allele.

Nucleic acid hybridization techniques are well known in the art. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

In some embodiments, probe molecules that hybridize to the mutant or wild type sequences can be used for detecting such sequences in the amplified product by solution phase or, more preferably, solid phase hybridization. Solid phase hybridization can be achieved, for example, by attaching probes to a microchip.

Nucleic acid probes may comprise ribonucleic acids and/or deoxyribonucleic acids. In some embodiments, provided nucleic acid probes are oligonucleotides (i.e., “oligonucleotide probes”). Generally, oligonucleotide probes are long enough to bind specifically to a homologous region of the gene of interest, but short enough such that a difference of one nucleotide between the probe and the nucleic acid sample being tested disrupts hybridization. Typically, the sizes of oligonucleotide probes vary from approximately 10 to 100 nucleotides. In some embodiments, oligonucleotide probes vary from 15 to 90, 15 to 80, 15 to 70, 15 to 60, 15 to 50, 15 to 40, 15 to 35, 15 to 30, 18 to 30, or 18 to 26 nucleotides in length. As appreciated by those of ordinary skill in the art, the optimal length of an oligonucleotide probe may depend on the particular methods and/or conditions in which the oligonucleotide probe may be employed.

In some embodiments, nucleic acid probes are useful as primers, e.g., for nucleic acid amplification and/or extension reactions. For example, in certain embodiments, the gene sequence being evaluated for a variant comprises the exon sequences. In certain embodiments, the exon sequence and additional flanking sequence (e.g., about 5, 10, 15, 20, 25, 30, 35, 40, 45, 50, 55 or more nucleotides of UTR and/or intron sequence) is analyzed in the assay. Or intron sequences or other non-coding regions may be evaluated for potentially deleterious mutations. Or portions of these sequences may be used. Such variant gene sequences may include sequences having at least one of the mutations as described herein.

Other embodiments of the disclosure provide isolated gene sequences containing mutations that relate to the syndrome and/or disease of interest. Such gene sequences may be used to objectively diagnose the presence or increased risk for a subject to develop HNSCC. In certain embodiments, the isolated nucleic acid may contain a non-variant sequence or a variant sequence of any one or combination thereof. For example, in certain embodiments, the gene sequence comprises the exon sequences. In certain embodiments, the exon sequence and additional flanking sequence (e.g., about 5, 10, 15, 20, 25, 30, 35, 40, 45, 50, 55 or more nucleotides of UTR and/or intron sequence) is analyzed in the assay. Or intron sequences or other non-coding regions may be used. Or portions of these sequences may be used. In certain embodiments, the gene sequence comprises an exon sequence from at least one of the biomarker genes disclosed herein.

In some embodiments, nucleic acid probes are labeled with a detectable moiety as described herein.

### Arrays

A variety of the methods mentioned herein may be adapted for use as arrays that allow sets of biomarkers to be analyzed and/or detected in a single experiment. For example, multiple mutations that comprise biomarkers can be analyzed at the same time. In particular, methods that involve use of nucleic acid reagents (e.g., probes, primers, oligonucleotides, etc.) are particularly amenable for adaptation to an array-based platform (e.g., microarray). In some embodiments, an array containing one or more probes specific for detecting mutations in the biomarker of interest.

In an embodiment, a panel of a plurality of the disclosed biomarkers are used. In an embodiment, the disclosure comprises a composition to detect biomarkers associated with Head and Neck Squamous Cell Carcinoma (HNSCC) in an individual comprising a reagent that quantifies the levels of expression of at least one of the genes in Tables 1, 2, 5 and/or 6, and/or at least one of AIM2, CDSN, INHBA, MMP1, MMP3, or MMP10. Additionally, the expression product of other genes including at least one of MMP13, CRISP3, MUC21, ADAM12, MMP3 or ISG15 may be measured. Or combinations of these genes (as disclosed herein) may be measured. Additionally and/or alternatively, the composition may include at least one normalization (e.g., housekeeping) gene. In an embodiment, the normalization gene may be KHDRBS1 and/or RPL30 or other normalization genes. The composition may, in certain embodiments, comprise primers and/or probes for any one of these genes, where the primers and/or probes are labeled with a detectable moiety as described herein.

### DNA Sequencing

In certain embodiments, diagnosis of the biomarker of interest is carried out by detecting variation in the sequence, genomic location or arrangement, and/or genomic copy number of a nucleic acid or a panel of nucleic acids by nucleic acid sequencing.

In some embodiments, the method may comprise obtaining a nucleic acid from a tissue or body fluid sample from a subject and sequencing at least a portion of a nucleic acid in order to obtain a sample nucleic acid sequence for at least one gene. In certain embodiments, the method may comprise comparing the variant to known variants associated with HNSCC and determining whether the variant is a variant that has been previously identified as being associated with HNSCC. Or the method may comprise identifying the variant as a new, previously uncharacterized variant. If the variant is a new variant, or in some cases for previously characterized (i.e., identified) variants, the method may further comprise performing an analysis to determine whether the mutation is expected to be deleterious to expression of the gene and/or the function of the protein encoded by the gene. The method may further comprise using the variant profile (i.e., a compilation of variants identified in the subject) to diagnose the presence of HNSCC or an increased risk of developing HNSCC.

For example, in certain embodiments, next generation (massively-parallel sequencing) may be used. Or Sanger sequencing may be used. Or a combination of next-generation (massively-parallel sequencing) and Sanger sequencing may be used. Additionally and/or alternatively, the sequencing comprises at least one of single-molecule sequencing-by-synthesis. Thus, in certain embodiments, a plurality of DNA samples are analyzed in a pool to identify samples that show a variation. Additionally and/or alternatively, in certain embodiments, a plurality of DNA samples are analyzed in a plurality of pools to identify an individual sample that shows the same variation in at least two pools.

One conventional method to perform sequencing is by chain termination and gel separation, as described by Sanger et al., 1977, Proc Natl Acad Sci USA, 74:5463-67. Another conventional sequencing method involves chemical degradation of nucleic acid fragments. See, Maxam et al., 1977, Proc. Natl. Acad. Sci., 74:560-564. Also, methods have been developed based upon sequencing by hybridization. See, e.g., Harris et al., U.S. Patent Application Publication No. 20090156412.

In other embodiments, sequencing of the nucleic acid is accomplished by massively parallel sequencing (also known as “next generation sequencing”) of single-molecules or groups of largely identical molecules derived from single molecules by amplification through a method such as PCR. Massively parallel sequencing is shown for example in Lapidus et al., U.S. Pat. No. 7,169,560, Quake et al. U.S. Pat. No. 6,818,395, Harris U.S. Pat. No. 7,282,337 and Braslavsky, et al., PNAS (USA), 100: 3960-3964 (2003).

In next generation sequencing, PCR or whole genome amplification can be performed on the nucleic acid in order to obtain a sufficient amount of nucleic acid for analysis. In some forms of next generation sequencing, no amplification is required because the method is capable of evaluating DNA sequences from unamplified DNA. Once determined, the sequence and/or genomic arrangement and/or genomic copy number of the nucleic acid from the test sample is compared to a standard reference derived from one or more individuals not known to suffer from HNSCC at the time their sample was taken. All differences between the sequence and/or genomic arrangement and/or genomic arrangement and/or copy number of the nucleic acid from the test sample and the standard reference are considered variants.

In next generation (massively parallel sequencing), all regions of interest are sequenced together, and the origin of each sequence read is determined by comparison (alignment) to a reference sequence. The regions of interest can be enriched together in one reaction, or they can be enriched separately and then combined before sequencing. In certain embodiments, and as described in more detail in the examples herein, the DNA sequences derived from coding exons of genes included in the assay are enriched by bulk hybridization of randomly fragmented genomic DNA to specific RNA probes. The same adapter sequences are attached to the ends of all fragments, allowing enrichment of all hybridization-captured fragments by PCR with one primer pair in one reaction. Regions that are less efficiently captured by hybridization are amplified by PCR with specific primers. In addition, PCR with specific primers is may be used to amplify exons for which similar sequences (“pseudo exons”) exist elsewhere in the genome.

In certain embodiments where massively parallel sequencing is used, PCR products are concatenated to form long stretches of DNA, which are sheared into short fragments (e.g., by acoustic energy). This step ensures that the fragment ends are distributed throughout the regions of interest. Subsequently, a stretch of dA nucleotides is added to the 3′ end of each fragment, which allows the fragments to bind to a planar surface coated with oligo(dT) primers (the “flow cell”). Each fragment may then be sequenced by extending the oligo(dT) primer with fluorescently-labeled nucleotides. During each sequencing cycle, only one type of nucleotide (A, G, T, or C) is added, and only one nucleotide is allowed to be incorporated through use of chain terminating nucleotides. For example, during the 1st sequencing cycle, a fluorescently labeled dCTP could be added. This nucleotide will only be incorporated into those growing complementary DNA strands that need a C as the next nucleotide. After each sequencing cycle, an image of the flow cell is taken to determine which fragment was extended. DNA strands that have incorporated a C will emit light, while DNA strands that have not incorporated a C will appear dark. Chain termination is reversed to make the growing DNA strands extendible again, and the process is repeated for a total of 120 cycles. The images are converted into strings of bases, commonly referred to as “reads,” which recapitulate the 3′ terminal 25 to 60 bases of each fragment. The reads are then compared to the reference sequence for the DNA that was analyzed. Since any given string of 25 bases typically only occurs once in the human genome, most reads can be “aligned” to one specific place in the human genome. Finally, a consensus sequence of each genomic region may be built from the available reads and compared to the exact sequence of the reference at that position. Any differences between the consensus sequence and the reference are called as sequence variants.

### Detectable Moieties

In certain embodiments, certain molecules (e.g., nucleic acid probes, antibodies, etc.) used in accordance with and/or provided by the invention comprise one or more detectable entities or moieties, i.e., such molecules are “labeled” with such entities or moieties.

Any of a wide variety of detectable agents can be used in the practice of the disclosure. Suitable detectable agents include, but are not limited to: various ligands, radionucleotides; fluorescent dyes; chemiluminescent agents (such as acridinium esters, stabilized dioxetanes, and the like); bioluminescent agents; spectrally resolvable inorganic fluorescent semiconductors nanocrystals (e.g., quantum dots); microparticles; metal nanoparticles (e.g., gold, silver, copper, platinum); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

In some embodiments, the detectable moiety is biotin. Biotin can be bound to avidins (such as streptavidin), which are typically conjugated (directly or indirectly) to other moieties (e.g., fluorescent moieties) that are detectable themselves.

Below are described some non-limiting examples of some detectable moieties that may be used.

### Fluorescent Dyes

In certain embodiments, a detectable moiety is a fluorescent dye. Numerous known fluorescent dyes of a wide variety of chemical structures and physical characteristics are suitable for use in the practice of the disclosure. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM), hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR)), coumarin and coumarin dyes (e.g., methoxycoumarin, dialkylaminocoumarin, hydroxycoumarin, aminomethylcoumarin (AMCA)), Q-DOTS, Oregon Green Dyes (e.g., Oregon Green 488, Oregon Green 500, Oregon Green 514), Texas Red, Texas Red-X, SPECTRUM RED, SPECTRUM GREEN, cyanine dyes (e.g., CY-3, CY-5, CY-3.5, CY5.5), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, OR. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92:4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described, for example, in U.S. Pat. No. 5,945,526 to Lee et al. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

### Enzymes

In certain embodiments, a detectable moiety is an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a molecule using a linker group such as a carbodiimide, a diisocyanate, a glutaraldehyde, and the like.

### Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (e.g., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201T1, 212Bi, 213Bi, 211At, 153Sm, 177Lu).

### Dendrimers

In some embodiments, signal amplification is achieved using labeled dendrimers as the detectable moiety (see, e.g., Physiol Genomics 3:93-99, 2000). Fluorescently labeled dendrimers are available from Genisphere (Montvale, N.J.). These may be chemically conjugated to the oligonucleotide primers by methods known in the art.

### Methods to Identify HNSCC Markers

### Data Mining

In certain embodiments of the disclosure, biomarkers are identified using a data mining approach. For example, in some cases public databases, e.g., PubMed, The Cancer Genome Atlas (TCGA) may be searched for genes that have been shown to be linked to (directly or indirectly) to a certain disease and/or differentially expressed in cancer as compared to normal tissue. Such genes may then be evaluated as biomarkers.

### Molecular

In certain embodiments, the disclosure comprises methods to identify biomarkers for a syndrome or disease of interest (i.e., variants in nucleic acid sequence that are associated with HNSCC in a statistically significant manner). For example, the genes of interest and potential normalization genes may be identified by evaluating gene expression in tissue samples isolated from patients that have head and neck cancer using Random Forest Analysis (see e.g., L. Breiman, “Random Forests” Machine Learning, 2001, 45:5-32) and as discussed in detail herein. In this approach, random forests are a combination of tree predictors such that each tree depends on the values of a random vector sampled independently and with the same distribution for all trees in the forest.

For example, as shown in commonly owned U.S. application Ser. No. 16/224,974, filed Dec. 19, 2018 and published as US 2019/0187143 A1 and incorporated by reference in its entirety herein, RNASeq dataset from head and neck cancer (HNC) and normal tissue samples in The Cancer Genome Atlas (TCGA) may be interrogated by Random Forest (RF) analysis to identify and rank differentially expressed genes that could be used as a diagnostic marker(s) to differentiate HNC from non-cancer samples. The RNASeq data may be filtered to include only those genes with a reported value for greater than 50% of the samples, and fold-change in expression greater than two with a Wilcox adjusted p-value less than 0.001. In an embodiment, seventy-five percent of the samples may be used for the training set and 25% for the samples for the test set. The results may be 10-fold cross validated, optimized for Cohen's kappa and the top 20 genes ranked. The entire process may be repeated multiple (e.g., four) times and the list of genes and rankings of each RF determined (see e.g., Table 3 of US 2019/0187143 A1) (in this table a rank of 20 was the highest, and 1 the lowest). A rank-sum of the genes from the four RF runs results in a list of 36 unique genes, as shown in Table 4 of US 2019/0187143 A1. The column entitled “No. of Times in R.F.” represents the number of times a particular gene appeared on a RF list. A gene appearing in all four RF repeats suggests that this may be a top candidate marker to differentiate HNC from non-cancer samples, and appear at the top of the list with the largest rank-sums.

As a complementary approach to RF analysis for the identification of differentially expressed genes, the TCGA HNC RNASeq dataset may be used to compare the % Overlap in Expression vs Fold-change in Expression (HNC/Normal).

For genes with an increased expression in HNC (up regulated) compared to normal tissue, the % Overlap in Expression may be defined as the percent of samples between the 95^{th} percentile of the normal distribution to the 5^{th} percentile of the HNC distribution (see e.g., FIG. 6 of US 2019/0187143 showing GRIN2D as an example). For genes with a decreased expression in HNC (down regulated) compared to normal tissue, the % Overlap in Expression may be defined as the percent of samples between the 95^{th} percentile of the HNC distribution to the 5^{th} percentile of the normal distribution. In certain embodiments, genes with a small % Overlap in Expression maybe better suited for use as a diagnostic marker(s) to differentiate HNC from non-cancer samples.

In some embodiments, median expression from the HNC samples may be divided by the median expression of the normal samples for each gene to determine Fold-change in Expression. In certain embodiments, genes with a large Fold-change in Expression may be better suited for use as a diagnostic marker(s) to differentiate HNC from non-cancer samples.

In certain embodiments, the top (e.g., about 10) genes identified from RF analysis may have less than 20% overlap in expression, further supporting the idea that genes with a small % Overlap in Expression maybe better suited for use as a diagnostic marker(s) to differentiate HNC from non-cancer samples, and also highlights the similarities between these two complementary approaches for the identification of differentially expressed genes.

Or, the genes and/or genomic regions assayed for new markers may be selected based upon their importance in biochemical pathways that show genetic linkage and/or biological causation to the syndrome and/or disease of interest. Or, the genes and/or genomic regions assayed for markers may be selected based on genetic linkage to DNA regions that are genetically linked to the inheritance of HNSCC in families. Or, the genes and/or genomic regions assayed for markers may be evaluated systematically to cover certain regions of chromosomes not yet evaluated.

In other embodiments, the genes or genomic regions evaluated for new markers may be part of a biochemical pathway that may be linked to the development of the syndrome and/or disease of interest (e.g., HNSCC). The variants and/or variant combinations may be assessed for their clinical significance based on one or more of the following methods. If a variant or a variant combination is reported or known to occur more often in nucleic acid from subjects with, than in subjects without, the syndrome and/or disease of interest it is considered to be at least potentially predisposing to the syndrome and/or disease of interest. If a variant or a variant combination is reported or known to be transmitted exclusively or preferentially to individuals having the syndrome and/or disease of interest, it is considered to be at least potentially predisposing to the syndrome and/or disease of interest. Conversely, if a variant is found in both populations at a similar frequency, it is less likely to be associated with the development of the syndrome and/or disease of interest.

If a variant or a variant combination is reported or known to have an overall deleterious effect on the function of a protein or a biological system in an experimental model system appropriate for measuring the function of this protein or this biological system, and if this variant or variant combination affects a gene or genes known to be associated with the syndrome and/or disease of interest, it is considered to be at least potentially predisposing to the syndrome and/or disease of interest. For example, if a variant or a variant combination is predicted to have an overall deleterious effect on a protein or gene expression (i.e., resulting in a nonsense mutation, a frameshift mutation, or a splice site mutation, or even a missense mutation), based on the predicted effect on the sequence and/or the structure of a protein or a nucleic acid, and if this variant or variant combination affects a gene or genes known to be associated with the syndrome and/or disease of interest, it is considered to be at least potentially predisposing to the syndrome and/or disease of interest.

Also, in certain embodiments, the overall number of variants may be important. If, in the test sample, a variant or several variants are detected that are, individually or in combination, assessed as at least probably associated with the syndrome and/or disease of interest, then the individual in whose genetic material this variant or these variants were detected can be diagnosed as being affected with or at high risk of developing the syndrome and/or disease of interest.

For example, the disclosure herein provides methods for diagnosing the presence or an increased risk of developing HNSCC in a subject. Such methods may include obtaining a nucleic acid from a sample of saliva from the subject. The method may comprise determining expression of at least one gene in both normal and cancer tissue to identify potential biomarkers of interest. The method may further include sequencing the nucleic acid or determining the genomic arrangement or copy number of the nucleic acid to detect whether there is a variant or variants in the nucleic acid sequence or genomic arrangement or copy number. The method may further include the steps of assessing the clinical significance of a variant or variants. Such analysis may include an evaluation of the extent of association of the variant sequence in affected populations (i.e., subjects having the disease). Such analysis may also include an analysis of the extent of the effect the mutation may have on gene expression and/or protein function. The method may also include diagnosing the presence or an increased risk of developing HNSCC based on the assessment.

### EXAMPLES

The following non-limiting examples serve to illustrate certain aspects of the invention.

### Example 1—Overview

The goal of this project was to develop a saliva-based screening test for cancers of the oral cavity and oropharynx. A simple collection device, such as one similar to the DNA Genotek CP-190, may be used to collect saliva during an annual physical exam with a primary care physician, a six-month preventive dental exam with a dentist, or for at home collection. The relative ease and noninvasive sample collection makes saliva an ideal bio-fluid. For screening of cancers of the head and neck, saliva may be a preferred sample, and may provide added sensitivity due to the direct contact with tissues of the oral cavity and oropharynx. FIG. 1 provides an illustration of the overall method in accordance with an embodiment of the disclosure.

### Example 2—Collection of Saliva, and Isolation and Quantification of RNA

Two milliliters (mLs) of saliva was collected from HNC patients and healthy volunteers in a DNA Genotek CP-190 collection device and mixed with two mLs of RNA stabilizing liquid. The samples can be stored at room temperature (RT) for up to 8 weeks, or ≤20° C. long term. Collection devices were shipped at ambient temperature, processed following manufacture instructions and stored at ≤70° C. FIG. 1 insert shows the range of sample volumes received for this study.

A 235 μL aliquot was removed from the saliva collection device and total RNA was isolated using the MagMax mirVanna™ Total RNA Isolation kit (ThermoFisher cat #A27828) on a KingFisher™ Flex Purification System. The MagMax mirVanna™ Total RNA Isolation kit includes GTC buffers and “silica-like” magnetic beads (Dynabeads™ MyOne™ Silane). The KingFisher™ Flex Purification System uses PK+DNase, with an eluate of about 50 μL.

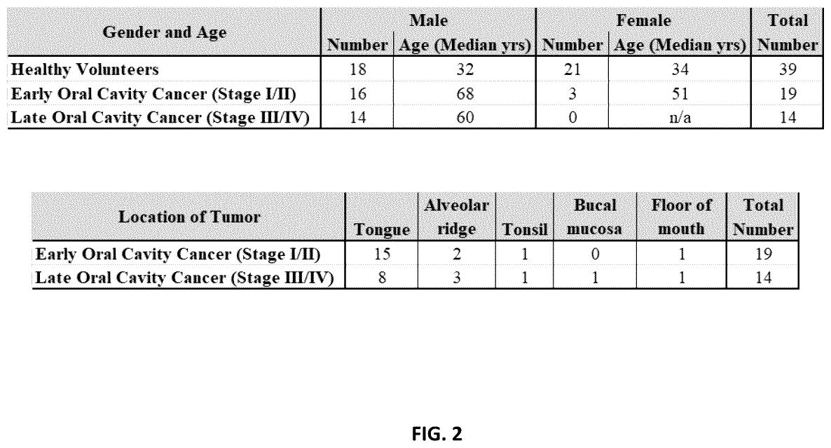

Eight μL of the 50 μL eluent from the KingFisher Flex was used to for the synthesis of first-strand cDNA with a SuperScript IV First-Strand Synthesis System Kit (ThermoFisher cat #18091050) using random hexamers following manufacturer recommended procedures. Twenty-three μL ddPCR reaction mixes were prepared using the Bio-Rad 2× ddPCR Supermix for Probes (No dUTP, Bio-Rad cat #1863024), 900 nM/250 nM target gene primers/probe (Bio-Rad ddPCR GEX FAM Assay, cat #10031252), and 900 nM/250 nM RPL30 (housekeeping gene) primers/probe (Bio-Rad ddPCR GEX HEX Assay, cat #10031255). Droplets were made in the Bio-Rad Automated Droplet Generator, and plates sealed with a pierceable foil using the Bio-Rad PCR Plate sealer. Thermal cycling was carried out in an Applied Biosystems Veriti 96-well Fast Thermal Cycler using the following conditions: 95° C. for 10 minutes (enzyme activation), 94° C. for 30 seconds and 55° C. for 1 min (annealing/extension) for 40 cycles, 98° C. for 10 minutes (enzyme deactivation), followed by a hold at 4° C. Droplets were detected on the Bio-Rad QX200 Droplet Reader and analyzed using the Bio-Rad QuantaSoft Analysis Pro Software, version 1.0, which reports units in copies/μL. FIG. 2 shows the gender, age, cancer stage, location of tumor and number of saliva samples that were used for this work.

### Example 3—Gene Selection in General

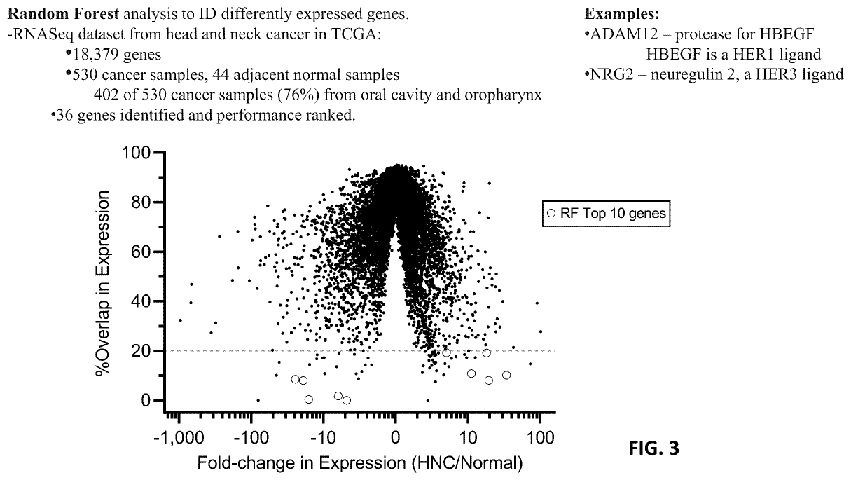

Candidate genes of interest were identified using a random forest approach as described in commonly owned U.S. application Ser. No. 16/224,974, filed Dec. 19, 2018 and published as US 2019/0187143 A1 (incorporated by reference in its entirety herein). For example, as shown in commonly owned US 2019/0187143 A1, RNASeq dataset from head and neck cancer (HNC) and normal tissue samples in The Cancer Genome Atlas (TCGA) was interrogated by Random Forest (RF) analysis to identify and rank differentially expressed genes that could be used as a diagnostic marker(s) to differentiate HNC from non-cancer samples. The RNASeq data was filtered to include only those genes with a reported value for greater than 50% of the samples, and fold-change in expression greater than two with a Wilcox adjusted p-value less than 0.001. Next, seventy-five percent of the samples were used for the training set and 25% for the samples for the test set. The results were 10-fold cross validated, optimized for Cohen's kappa and the top 20 genes ranked. The entire process was repeated four times and the list of genes and rankings of each RF determined (see e.g., Table 3 of co-owned U.S. Patent Publication No. US 2019/0187143 showing a gene ranking where 20 is the highest and 1 the lowest). A rank-sum of the genes from the four RF runs resulted in a list of 36 unique genes, as shown in FIG. 3 (from U.S. application Ser. No. 16/224,974, filed Dec. 19, 2018 and published as US 2019/0187143 A1). Modified rankings developed under this analysis are shown in Table 1 below. The column entitled “No. of Times in R.F.” represents the number of times a particular gene appeared on a RF list. A gene appearing in all four RF repeats suggested that this may be a top candidate marker to differentiate HINC from non-cancer samples; such genes appear at the top of the list with the largest rank-sums.

TABLE 1

| | | Median | | Median | % Overlap | No. of | |
|---|---|---|---|---|---|---|---|
| Rank | | Expression (log₂) | | Fold- | in | Times | |
| Sum | Gene Symbol\|ID | Normal | HNSCC | Change | Expression | in RF | Full Name of Gene |
| 72 | SH3BGRL2\|83699 | 11.44 | 7.44 | −16 | 0% | 4 | SH3 domain binding |
| | | | | | | | glutamate rich protein like 2 |
| 69 | CAB39L\|81617 | 9.14 | 6.89 | −5 | 0% | 4 | Calcium-binding protein |
| | | | | | | | 39-like |
| 58 | HSD17B6\|8630 | 2.99 | 5.33 | 5 | 19% | 4 | Hydroxysteroid 17-beta |
| | | | | | | | dehydrogenase 6 |
| 57 | NRG2\|9542 | 5.75 | 1.50 | −19 | 8% | 4 | Neuregulin 2 |
| 52 | GRIN2D\|2906 | 3.70 | 7.19 | 11 | 11% | 4 | Glutamate [NMDA] |
| | | | | | | | receptor subunit epsilon-4 |
| 47 | MMP11\|4320 | 5.68 | 10.78 | 34 | 10% | 4 | Matrix metalloproteinase- |
| | | | | | | | 11 |
| 46 | GPD1L\|23171 | 10.88 | 8.24 | −6 | 2% | 4 | Glycerol-3-phosphate |
| | | | | | | | dehydrogenase 1 like |
| 39 | DLG2\|1740 | 6.92 | 2.30 | −25 | 9% | 4 | Disks large homolog 2, also |
| | | | | | | | known as channel- |
| | | | | | | | associated protein of |
| | | | | | | | synapse-110 (chapsyn-110) |
| | | | | | | | or postsynaptic density |
| | | | | | | | protein 93 (PSD-93) |
| 38 | ADAM12\|8038 | 5.33 | 9.52 | 18 | 19% | 4 | Disintegrin and |
| | | | | | | | metalloproteinase domain- |
| | | | | | | | containing protein 12 |
| 35 | IL11\|3589 | 2.55 | 6.84 | 19 | 8% | 3 | Interleukin 11 |
| 33 | GPRIN1\|114787 | 6.55 | 8.81 | 5 | 15% | 3 | G protein-regulated inducer |
| | | | | | | | of neurite outgrowth 1 |
| 28 | TMEM132C\|92293 | 5.46 | 1.18 | −19 | 45% | 2 | Transmembrane Protein |
| | | | | | | | 1320 |
| 27 | MGC12982\|84793 | 3.71 | 5.91 | 5 | 10% | 2 | FOXD2 adjacent opposite |
| | | | | | | | strand RNA 1 |
| 21 | COL13A1\|1305 | 3.48 | 6.26 | 7 | 18% | 2 | Collagen alpha-1(XIII) |
| | | | | | | | chain |
| 21 | KRT4\|3851 | 18.41 | 8.99 | −685 | 39% | 2 | Keratin, type I cytoskeletal |
| | | | | | | | 4 |
| 19 | RRAGD\|58528 | 10.61 | 8.00 | −6 | 19% | 2 | Ras-related GTP-binding |
| | | | | | | | protein D |
| 18 | LOXL2\|4017 | 7.15 | 10.42 | 10 | 17% | 1 | Lysyl oxidase homolog 2 |
| 17 | ESM1\|11082 | 2.98 | 6.79 | 14 | 55% | 2 | Endothelial cell-specific |
| | | | | | | | molecule 1 |
| 16 | FAM107A\|11170 | 9.23 | 5.28 | −16 | 14% | 2 | Family with sequence |
| | | | | | | | similarity 107 member A |
| 15 | GCOM1\|145781 | 9.72 | 6.43 | −10 | 17% | 1 | GRINL1A combined |
| | | | | | | | protein 15 |
| 15 | SHROOM3\|57619 | 10.41 | 8.50 | −4 | 39% | 2 | Shroom-related protein 3 |
| 14 | MUC21\|394263 | 14.43 | 4.53 | −956 | 32% | 2 | Mucin 21 |
| 11 | COBL\|23242 | 9.97 | 6.71 | −10 | 11% | 1 | Cordon-bleu protein (Cobl) |
| | | | | | | | is an actin nucleator protein |
| 11 | EMP1\|2012 | 15.70 | 12.45 | −9 | 54% | 1 | Epithelial membrane |
| | | | | | | | protein 1 |
| 11 | MAL\|4118 | 14.42 | 6.14 | −309 | 31% | 2 | Myelin and lymphocyte |
| | | | | | | | protein |
| 9 | ATP6V0A4\|50617 | 9.11 | 4.48 | −25 | 29% | 1 | V-type proton ATPase 116 |
| | | | | | | | kDa subunit a isoform 4 |
| 6 | AQP7\|364 | 5.17 | 1.13 | −17 | 25% | 1 | Aquaporin-7 |
| 6 | BARX2\|8538 | 11.65 | 9.01 | −6 | 28% | 1 | BARX homeobox 2 |
| 6 | FAM3D\|131177 | 11.45 | 5.97 | −45 | 10% | 3 | Family with sequence |
| | | | | | | | similarity 3, member D |
| 5 | MMP9\|4318 | 7.04 | 11.19 | 18 | 22% | 2 | Matrix metalloproteinase-9 |
| 5 | MYBL2\|4605 | 9.03 | 10.86 | 4 | 8% | 1 | Myb-related protein B |
| 4 | CRISP3\|10321 | 11.63 | 3.15 | −357 | 27% | 1 | Cysteine-rich secretory |
| | | | | | | | protein 3 |
| 3 | CAMK2N2\|94032 | 2.70 | 5.15 | 5 | 14% | 1 | Calcium/calmodulin |
| | | | | | | | dependent protein kinase II |
| | | | | | | | inhibitor 2 |
| 2 | ADH1B\|125 | 8.66 | 2.34 | −80 | 0% | 1 | Alcohol dehydrogenase 1B |
| 2 | GPD1\|2819 | 8.20 | 3.20 | −32 | 24% | 1 | Glycerol-3-phosphate |
| | | | | | | | dehydrogenase |
| 2 | NDRG2\|57447 | 12.94 | 10.23 | −7 | 14% | 1 | NMYC downstrean- |
| | | | | | | | regulated gene 2 |

As a complementary approach to RF analysis for the identification of differentially expressed genes, the TCGA HINC RNASeq dataset may be used to compare the % Overlap in Expression vs Fold-change in Expression (HINC/Normal).

For genes with an increased expression in HNC (up regulated) compared to normal tissue, the % Overlap in Expression may be defined as the percent of samples between the 95^{th} percentile of the normal distribution to the 5^{th} percentile of the HINC distribution (see e.g., FIG. 6 in commonly owned U.S. Patent Publication No. US 2019/0187143). For genes with a decreased expression in HINC (down regulated) compared to normal tissue, the % Overlap in Expression may be defined as the percent of samples between the 95^{th} percentile of the HINC distribution to the 5^{th} percentile of the normal distribution. In certain embodiments, genes with a small % Overlap in Expression maybe better suited for use as a diagnostic marker(s) to differentiate HNC from non-cancer samples.

In some cases, median expression from the HNC samples was divided by the median expression of the normal samples for each gene to determine Fold-change in Expression. Genes with a large Fold-change in Expression may be better suited for use as a diagnostic marker(s) to differentiate HNC from non-cancer samples.

The top (e.g., about 10) genes identified from RF analysis shown as open circles in FIG. 3 and Table 1, SH3BGRL2, CAB39L, HSD17B6, NRG2, GRIN2D, MMP11, GPD1L, DLG2, ADAM12, and IL11, were found to have less than 20% overlap in expression, further supporting the idea that genes with a small % Overlap in Expression maybe better suited for use as a diagnostic marker(s) to differentiate HNC from non-cancer samples, and also highlights the similarities between these two complementary approaches for the identification of differentially expressed genes.

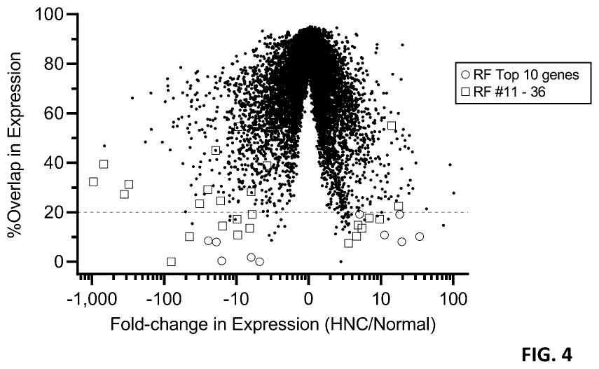

The remaining genes identified from RF analysis (#11-36) are shown (as open squares) in the representation of the data shown in FIG. 4 (from U.S. application Ser. No. 16/224,974, filed Dec. 19, 2018 and published as US 2019/0187143 A1). In total, 23 of the 36 (64%) genes identified by RF analysis have less than 20% overlap in expression.

A potential advantage of the graphical representation was the identification of additional genes not selected by RF analysis, in particular 45 genes with less than or equal to 20% overlap in expression These 45 genes are listed in Table 2 (also shown as Table 6 in U.S. Patent Publication No. US 2019/0187143). These are shown as solid black circles below the 20% overlap line in FIG. 3 and FIG. 4. Similar to the genes identified by RF analysis, the expression of these additional genes with less than 20% overlap in expression may be useful as a diagnostic marker(s) to differentiate HNC from non-cancer samples.

TABLE 2

| | | Median | | Median | % Overlap | |
|---|---|---|---|---|---|---|
| | | Expression (log₂) | | Fold- | in | |
| No. | Gene Symbol\|ID | Normal | HNSCC | change | Expression | Full Name of Gene |
| 1 | GLT25D1\|79709 | 10.544 | 12.029 | 3 | 0% | Collagen beta(1-O) |
| | | | | | | galactosyltransferase 1 |
| 2 | ARHGEF10L\|55160 | 11.027 | 9.326 | −3 | 9% | Rho guanine nucleotide exchange |
| | | | | | | factor 12 |
| 3 | PAIP2B\|400961 | 9.139 | 7.322 | −4 | 11% | Poly(A)-binding |
| | | | | | | protein interacting protein 2B |
| 4 | C20orf20\|55257 | 8.139 | 9.375 | 2 | 11% | MRG domain binding protein |
| 5 | UBL3\|5412 | 10.886 | 9.334 | −3 | 12% | Ubiquitin-like protein 3 |
| 6 | CDCA5\|113130 | 8.235 | 10.115 | 4 | 12% | Sororin |
| 7 | CDH24\|64403 | 6.547 | 8.120 | 3 | 13% | Cadherin 24 |
| 8 | RFC4\|5984 | 7.697 | 9.271 | 3 | 13% | Replication factor C subunit 4 |
| 9 | CENPO\|79172 | 6.467 | 7.742 | 2 | 14% | Centromere protein O |
| 10 | Clorf135\|79000 | 5.457 | 7.120 | 3 | 14% | Aurora kinase A and ninein interacting |
| | | | | | | protein |
| 11 | SUCLG2\|8801 | 10.225 | 9.123 | −2 | 14% | Succinate-CoA ligase GDP-forming |
| | | | | | | beta subunit |
| 12 | ETFDH\|2110 | 9.793 | 8.398 | −3 | 14% | Electron transfer flavoprotein |
| | | | | | | dehydrogenase |
| 13 | CA9\|768 | 2.720 | 8.917 | 73 | 15% | Carbonic anhydrase 9 |
| 14 | C16orf59\|80178 | 5.608 | 7.419 | 4 | 15% | Tubulin epsilon and delta complex 2 |
| 15 | KIF2C\|11004 | 8.238 | 9.996 | 3 | 15% | Kinesin family member 2C |
| 16 | EME1\|146956 | 4.893 | 6.749 | 4 | 15% | Essential meiotic structure-specific |
| | | | | | | endonuclease 1 |
| 17 | FMO2\|2327 | 11.440 | 6.084 | −41 | 15% | Flavin containing monooxygenase 2 |
| 18 | TGFB1\|7040 | 10.028 | 11.565 | 3 | 16% | Transforming growth factor beta 1 |
| 19 | FOXM1\|2305 | 9.260 | 10.972 | 3 | 16% | Forkhead box M1 |
| 20 | CGNL1\|84952 | 10.117 | 6.485 | −12 | 17% | Cingulin like 1 |
| 21 | BMP8A\|353500 | 4.361 | 6.935 | 6 | 17% | Bone morphogenetic protein 8a |
| 22 | ALDH9A1\|223 | 11.965 | 10.696 | −2 | 17% | Aldehyde dehydrogenase 9 family |
| | | | | | | member A1 |
| 23 | ASPA\|443 | 4.172 | 0.804 | −10 | 17% | Aspartoacylase |
| 24 | LAMC2\|3918 | 10.855 | 14.522 | 13 | 17% | Laminin subunit gamma 2 |
| 25 | CEP55\|55165 | 8.385 | 9.975 | 3 | 18% | Centrosomal protein 55 |
| 26 | AURKA\|6790 | 7.658 | 9.451 | 3 | 18% | Aurora kinase A |
| 27 | E2F1\|1869 | 7.017 | 8.661 | 3 | 18% | E2F transcription factor 1 |
| 28 | TPX2\|22974 | 9.740 | 11.276 | 3 | 18% | TPX2, microtubule nucleation factor |
| 29 | SLC27A6\|28965 | 7.097 | 1.560 | −46 | 18% | Solute carrier family 27 member 6 |
| 30 | LEPRE1\|64175 | 7.956 | 9.806 | 4 | 18% | Proly1 3-hydroxylase 1 |
| 31 | RORC\|6097 | 8.848 | 5.241 | −12 | 18% | RAR related orphan receptor C |
| 32 | MFAP2\|4237 | 7.267 | 10.430 | 9 | 18% | Microfibril associated protein 2 |
| 33 | NFIX\|4784 | 12.267 | 10.367 | −4 | 19% | Nuclear factor I X |
| 34 | PKMYT1\|9088 | 7.884 | 9.705 | 4 | 19% | Protein kinase, membrane associated |
| | | | | | | tyrosine/threonine 1 |
| 35 | VAV2\|7410 | 8.904 | 10.588 | 3 | 19% | Vav guanine nucleotide exchange |
| | | | | | | factor 2 |
| 36 | CENPA\|1058 | 6.188 | 7.972 | 3 | 19% | Centromere protein A |
| 37 | NETO2\|81831 | 7.647 | 9.616 | 4 | 19% | Neuropilin and tolloid like 2 |
| 38 | UBE2C\|11065 | 8.350 | 10.050 | 3 | 19% | Ubiquitin conjugating enzyme E2 C |
| 39 | C11orf84\|144097 | 7.680 | 9.029 | 3 | 20% | Spindlin interactor and repressor of |
| | | | | | | chromatin binding |
| 40 | FAM63A\|55793 | 9.550 | 8.068 | −3 | 20% | MINDY lysine 48 deubiquitinase 1 |
| 41 | WISP1\|8840 | 3.827 | 7.180 | 10 | 20% | Cellular communication network |
| | | | | | | factor 4 |
| 42 | BMP1\|649 | 9.031 | 10.846 | 4 | 20% | Bone morphogenetic protein 1 |
| 43 | PLIN1\|5346 | 6.879 | 1.220 | −51 | 20% | Perilipin 1 |
| 44 | KAT2B\|8850 | 10.368 | 8.285 | −4 | 20% | Lysine acetyltransferase 2B |
| 45 | CYP2J2\|1573 | 8.318 | 6.432 | −4 | 20% | Cytochrome P450 family 2 subfamily |
| | | | | | | J member 2 |

### Example 4—Gene Selection in Saliva

The genes that had their expression levels measured from saliva from HNC and healthy volunteers were identified. It was found that there are 26 genes that have had their expression levels measured by ddPCR from the saliva of HNC and healthy volunteers.

The initial search for saliva biomarkers began with genes that have a >10 fold-change in expression in the TCGA HNC RNASeq dataset. Thus, the TCGA HNC RNASeq dataset, which was derived from either HNC or normal tissue, was used as a predictive model system for saliva, where saliva from a HNC patient is a mixture of RNA transcripts from both cancerous and normal tissues present in the oral cavity, starting with genes that have relatively large, >10 fold-change in expression in the TCGA HNC RNASeq dataset to improve the likelihood of finding genes with a change in expression in saliva.

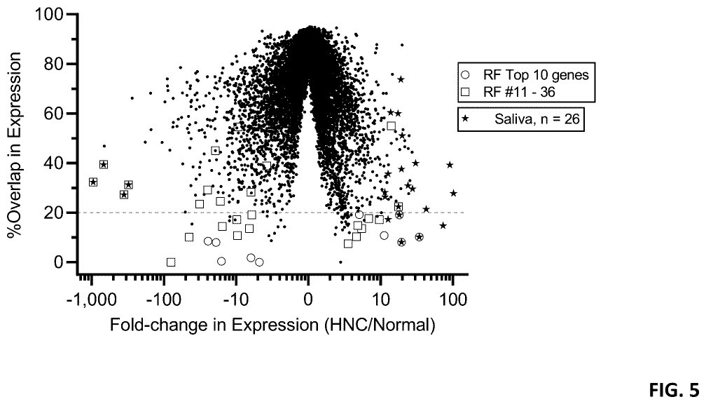

As can be seen in FIG. 5, twenty-two of the 26 genes identified using the TCGA HNC RNASeq dataset were up-regulated (a positive HNC/Normal quotient) and four of the 26 were down-regulated (a negative HNC/Normal quotient) in the TCGA HNC RNASeq dataset.

### Example 5—Saliva-Duplex ddPCR

The platform used to measure gene expression from saliva was the Bio-Rad droplet digital PCR (ddPCR). The Bio-Rad QX200 Droplet Reader is capable of identifying two colors, so duplex ddPCR reactions were performed to measure both the gene of interest (i.e., the candidate biomarker) labeled with FAM and a housekeeping gene (RPL30) labeled with HEX together in one ddPCR reaction. The results are shown in FIG. 6.

The graph on the top of FIG. 6 shows the level of gene expression in copies/μL (y-axis) for each saliva sample for the 26 genes measured (x-axis). The dotted line across the bottom of both graphs labeled as “No Call” represents a ddPCR result from a saliva sample with too few positive droplets obtain the copies/μL. The genes are sorted from low to high median expression. There was >18,000-fold range of expression, from 0.12 to 2,279 copies/μL, with median expression ranging nearly 300-fold, from 0.35 to 102 copies/μL.

The graph on the bottom of FIG. 6 shows the level of RPL30 (a housekeeping gene) expression in copies/μL (y-axis) for each saliva sample from the 26 genes analyzed (x-axis). In contrast to the distributions from the expression of the individual genes above, the distributions of RPL30 expression are not significantly different (Kruskal-Wallis p=0.2451).

Normalized ddPCR was the resulting quotient from dividing the gene copies/μL by the RPL30 copies/μL for each sample: Normalized ddPCR=(Gene-FAM copies/μL)/(RPL20-HEX copies/μL)

### Example 6—Median Normal Expression

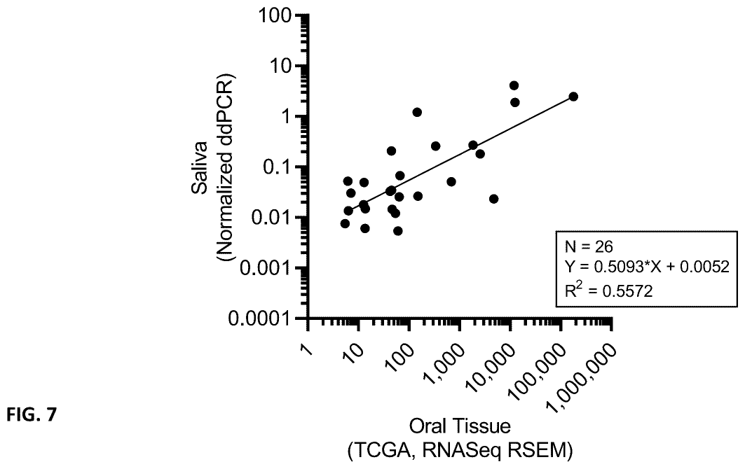

Median normal expression from the 26 genes was compared from the saliva normalized ddPCR results from healthy volunteers to what was reported in the TCGA HNC RNASeq dataset from normal tissue samples. As shown in FIG. 7, there appeared to be a weak, positive relationship (R²=0.5572) between gene expression measured from saliva by ddPCR and gene expression measured from oral tissue by RNASeq.

However, as noted in Table 3, there was a large difference in the range of expression (equal to the maximum expression/minimum expression) between the two measurements. From oral tissue reported in the TCGA HNC RNASeq dataset, the range in expression was 32,734-fold, while the range in expression from the same genes measured in saliva via ddPCR was 758-fold, a reduction of >43-fold. These large differences in expression may be attributed to the fact that the TCGA HNC RNASeq dataset was derived from either HNC or normal tissue, whereas saliva from a HNC patient is composed of a mixture of RNA transcripts from both cancerous and normal tissues present in the oral cavity. Despite these limitations, the utilization of the TCGA HNC RNASeq dataset provides utility for predicting gene expression levels in saliva.

TABLE 3

| Sample Type | Max | Min | Max/Min |
|---|---|---|---|
| Oral Tissue | 179,061 | 5.47 | 32,734 |
| Saliva | 4.12 | 0.005 | 758 |

### Example 7—Median Fold-Change in Expression

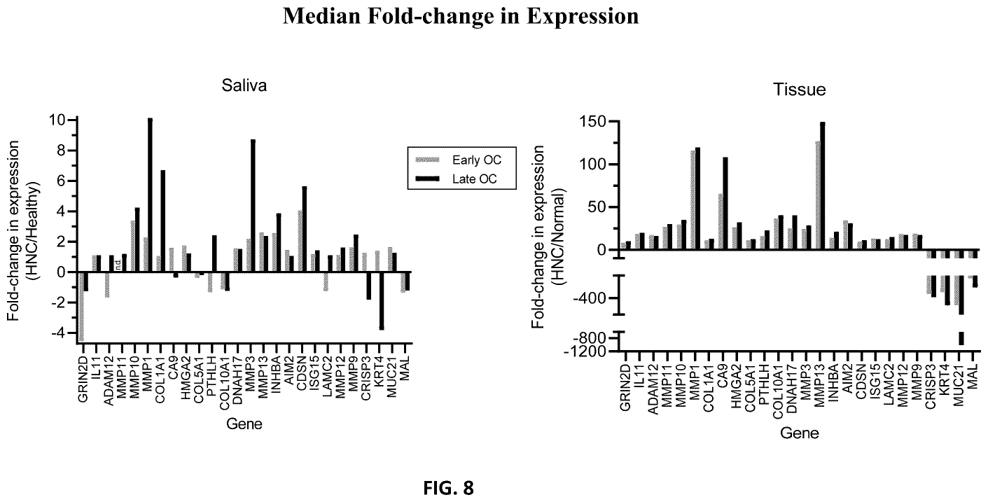

FIG. 8 shows median fold-change in expression as calculated separately for early oral cavity cancer (Early OC, stage I/II) and late oral cavity cancer (Late OC, stage III/IV) for both measured saliva samples, left graph, and the TCGA HNC RNASeq dataset (tissue), right graph for each of the 26 genes.

Many of the genes measured in saliva resulted in a small, +/−2-fold or less change in expression. Interestingly, the median fold-change in expression from a few genes (e.g., MMP1, COL1A1, MMP3, GRIN2D and KRT4) was much larger from late OC compared to early OC. The increase in fold-change in expression may not be surprising since the late OC sample are from a more advanced stage of cancer than the early OC samples, and possibly represent a larger tumor or multiple sites with cancerous tissue. More importantly, the increase in gene expression observed from the saliva of the late stage compared to the early stage cancer patients supports a relationship between these genes and oral cancer.

In contrast to the fold-changes observed from saliva, the fold-change in expression from TCGA HNC RNASeq dataset for tissues samples were much larger, up to 149-fold for genes that were upregulated to −1,006-fold for genes that were downregulated. Of note, the four genes on the far right of the graph that are all downregulated (CRISP3, KRT4, MUCH and MAL) in the TCGA HNC RNASeq dataset, only one of the four (MAL) was downregulated in early OC from saliva. Even though TCGA HNC RNASeq dataset suggests relatively large reductions in expression (−139 to −1,006-fold) for these four genes, a reduction in expression was not readily detected from the same genes in saliva. Again, these differences may be attributed to the TCGA HNC RNASeq dataset was derived from either HNC or normal tissue, whereas saliva from a HNC patient is composed of a mixture of RNA transcripts from both cancerous and normal tissues present in the oral cavity.

### Example 8—Normalized ddPCR at low RPL30

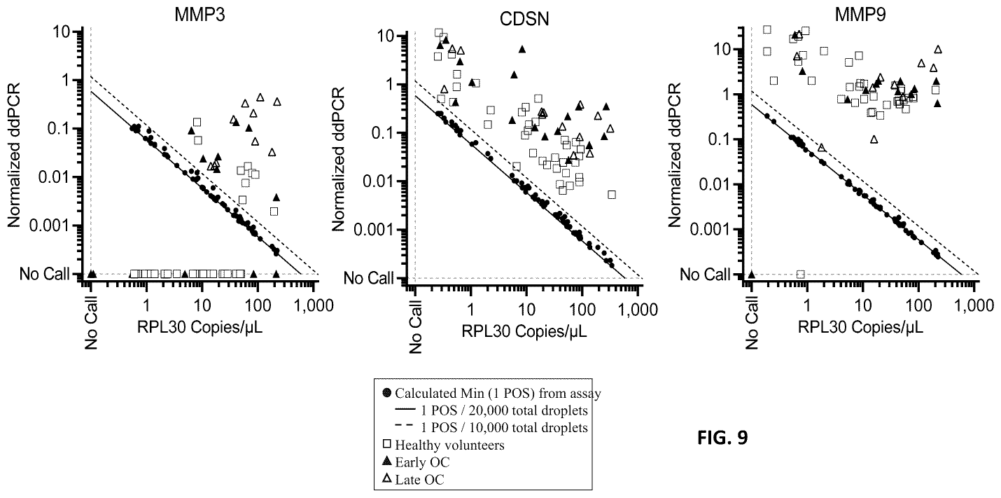

One goal of normalizing gene expression data is to reduce technical variation while preserving biological variation, and plotting the normalized ddPCR vs the RPL30 copies/μL was an attempt to evaluate data normalization. Results are shown in FIG. 9.

As shown in FIG. 9, the solid diagonal line was the calculated copies/μL from 1 positive droplet out of 20,000 total droplets (the intended number of total droplets), the dashed diagonal line was the calculated copies/μL from 1 positive droplet out of 10,000 total droplets (the minimal number of total droplets acceptable for a copies/μL calculation by the QantaSoft software) and the solid black dots are the calculated copies/μL from 1 positive droplet out of the actual number of droplets in the assay well. Assay wells with the total number of droplets closer to 20,000 was preferred, as the total number of droplets in an assay well increases the lower limit-of-detection improves or decreases.

Three of the 26 genes shown are representative of low (MMP3), medium (CDSN) and high (MMP9) gene expression levels in saliva. Many samples, and from a wide range of RPL30 copies/μL, resulted in a “No Call” from many genes, e.g., MMP3, GRIN2D, HMGA2, COL5A1, and MMPP12 (see FIG. 6) with a low expression levels. While there was a weak, positive relationship observed between median normal gene expression from the TCGA HNC RNASeq dataset and gene expression measured from saliva by ddPCR (FIG. 7), the detection limit of gene expression from saliva was evaluated empirically by ddPCR.

Normalized ddPCR as shown in FIG. 9 was the quotient of the gene copies/μL divided by the RPL30 copies/μL. The normalized ddPCR result from genes with medium (CDSN) and high (MMP9) expression levels were largest with small RPL30 copies/μL, suggestive of over-normalization following division with a small RPL30 copies/μL. To minimize over-normalization, normalized ddPCR values that were obtained with an RPL30 copies/μL of 2 or lower were removed (see Example 8).

### Example 9—RPL30 cutoff

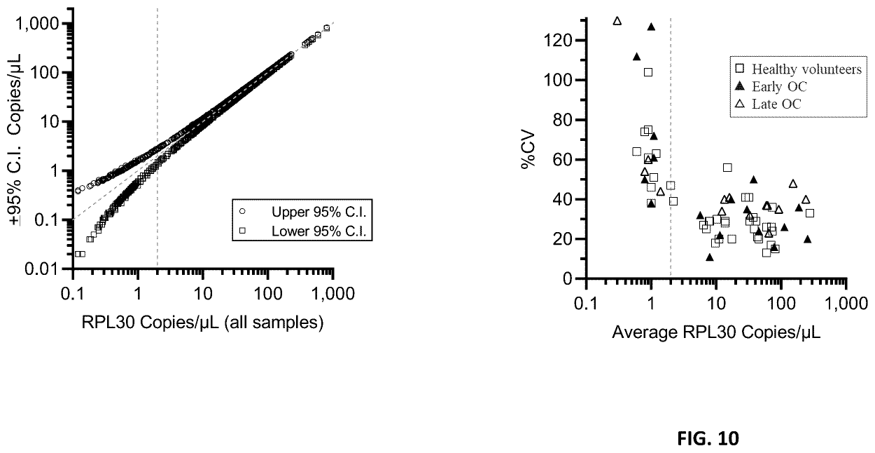

A RPL30 cutoff at ≤2 copies/μL was established to minimize over-normalization due to small RPL30 copies/μL. Results are shown in FIG. 10.

Thus, as shown in FIG. 10, the graph on the left represents the upper and lower 95% confidence intervals (C.I.) for each RPL30 copies/μL from all samples. As can been seen in the graph and Table 4 below, as the RPL30 copies/μL decreased, the range between the upper and lower 95% C.I. increased. To aid in the detection of small changes in gene expression, a precise measurement was preferred, so a RPL30 cut-off with a 2-fold range in C.I. was selected, which corresponded to 2-copies/μL. At RPL30>2 copies/μL, the 95% confidence interval range is <2 fold.

TABLE 4

| RPL30 | 95% Confidence Intervals | | |
|---|---|---|---|
| Copies/μL | Upper | Lower | Upper/Lower |
| 12 | 13.7 | 10.3 | 1.3 |
| 6 | 7.3 | 4.8 | 1.5 |
| 3 | 3.9 | 2.2 | 1.8 |
| 2 | 2.8 | 1.4 | 2.0 |
| 1 | 1.6 | 0.6 | 2.7 |
| 0.5 | 0.9 | 0.2 | 4.3 |

In FIG. 10, the graph on the right compares the percent coefficient of variation (% CV) to the RPL30 copies/μL from all samples. As the average RPL30 copies/μL decreased there was a dramatic increase in the variability of the measurement, up to 130% CV at 0.3 copies/μL. In contrast, samples with an RPL30 of >2 copies/μL (dashed vertical line), the median CV was 28%, with CV's ranging from 11 to 56%. Based on the wide C.I. and increasing % CV, samples with less than or equal to RPL30 of 2 copies/μL were excluded from further analysis. In summary, at RPL30>2 copies/μL, the 95% confidence interval range is <2 fold, and the median % CV is 28% (range 11-56%).

### Example 10—Differences Between Healthy Volunteers and Early OC

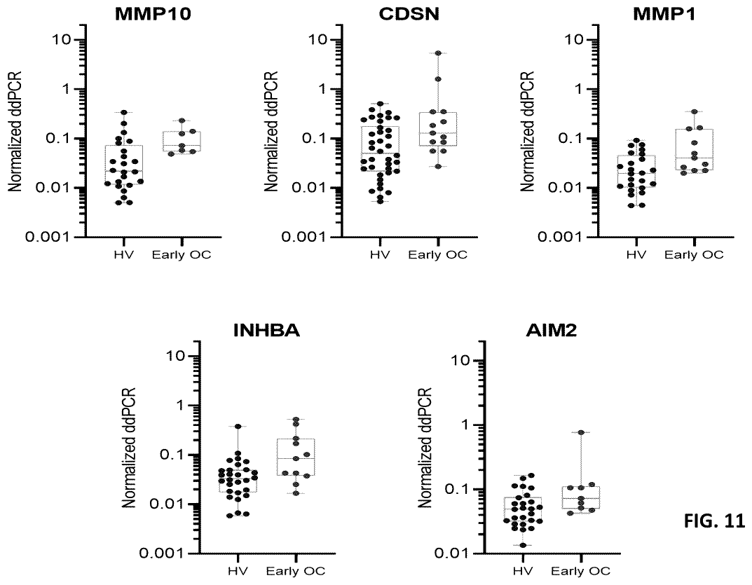

Statistical differences between normalized ddPCR expression levels from the saliva of healthy volunteers compared to the saliva from early oral cavity (OC) cancer patients for all genes was evaluated using an unpaired t-test and Wilcox Rank Sum Test. Results are shown in FIG. 11 and Table 5. In Table 5, genes are listed from smallest to largest p value from the t-test. P values were also adjusted for multiple testing using the false discovery rate and Bonferroni correction methods. A p value less than 0.05 was considered statistically significant. Not applicable (NA) designates genes with insufficient sample numbers for comparison.

TABLE 5

| | Number of | | | | | | | |
|---|---|---|---|---|---|---|---|---|
| | Samples | | t test | | | Wilcox Rank Sum Test | | |
| | | Early | | Adjusted p value | | | Adjusted p value | |
| Gene | HV | OC | p value | FDR | Bonferroni | p value | FDR | Bonferroni |
| MMP10 | 20 | 6 | 0.0014 | 0.0274 | 0.0274 | 0.0194 | 0.1135 | 0.3878 |
| CDSN | 29 | 13 | 0.0041 | 0.0407 | 0.0815 | 0.0034 | 0.0689 | 0.0689 |
| MMP1 | 25 | 11 | 0.0109 | 0.0724 | 0.2171 | 0.0170 | 0.1135 | 0.3398 |
| INHBA | 28 | 11 | 0.0195 | 0.0977 | 0.3906 | 0.0227 | 0.1135 | 0.4541 |
| AIM2 | 20 | 9 | 0.0386 | 0.1545 | 0.7724 | 0.0593 | 0.2373 | 1 |
| MMP13 | 9 | 4 | 0.1038 | 0.3349 | 1 | 0.1986 | 0.4413 | 1 |
| CRISP3 | 14 | 6 | 0.1300 | 0.3349 | 1 | 0.0913 | 0.2609 | 1 |
| MUC21 | 11 | 6 | 0.1340 | 0.3349 | 1 | 0.1215 | 0.3038 | 1 |
| ADAM12 | 27 | 10 | 0.2539 | 0.5100 | 1 | 0.2449 | 0.4453 | 1 |
| MMP9 | 28 | 11 | 0.2550 | 0.5100 | 1 | 0.0861 | 0.2609 | 1 |
| MMP3 | 9 | 6 | 0.2914 | 0.5186 | 1 | 0.2238 | 0.4453 | 1 |
| ISG15 | 26 | 11 | 0.3111 | 0.5186 | 1 | 0.3521 | 0.5869 | 1 |
| KRT4 | 13 | 6 | 0.6662 | 1 | 1 | 0.7012 | 1 | 1 |
| COL10A1 | 29 | 12 | 0.7839 | 1 | 1 | 0.8635 | 1 | 1 |
| PTHLH | 29 | 12 | 0.8384 | 1 | 1 | 0.7745 | 1 | 1 |
| LAMC2 | 26 | 11 | 0.9229 | 1 | 1 | 1 | 1 | 1 |
| COL1A1 | 27 | 10 | 0.9480 | 1 | 1 | 0.8912 | 1 | 1 |
| CA9 | 2 | 1 | NA | NA | NA | NA | NA | NA |
| COL5A1 | 1 | 1 | NA | NA | NA | NA | NA | NA |
| MMP11 | 1 | 0 | NA | NA | NA | NA | NA | NA |

Distributions of the normalized ddPCR results from healthy volunteers (HV) and early oral cavity (OC) cancer patients for five genes are also shown (FIG. 11). Significantly higher levels of expression of AIM2, CDSN, INHBA, MMP1, and MMP10 (t-test p<0.05) were found in the saliva of early OC patients compared to the saliva from healthy volunteers.

### Example 11—ROC Analysis

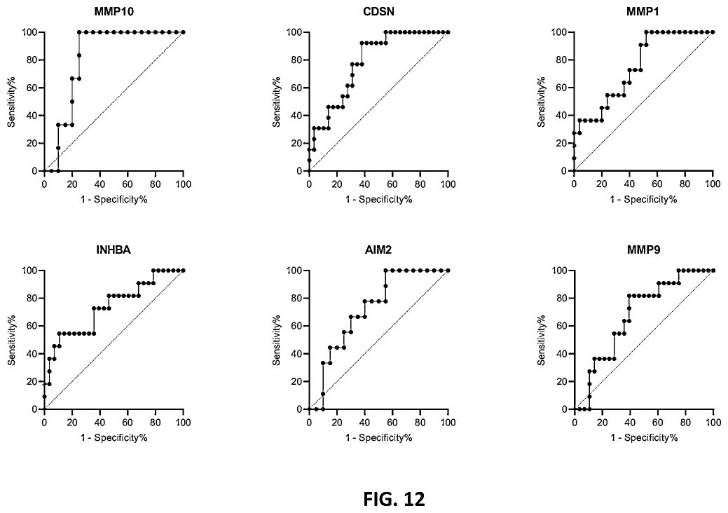

The performance of the gene expression levels to classify a saliva sample from either a healthy volunteer or from an early oral cavity cancer patient was evaluated by Receiver Operator Characteristic (ROC) analysis. Results are shown in FIG. 12 and Table 6. In Table 6, genes were listed from largest to smallest area under the curve (AUC), together with the associated performance metrics at the optimal cut-off. AUCs with significant p values (p<0.05) are shown in bold font. NA=genes with insufficient sample numbers for evaluation.

TABLE 6

| | Number of | | | | | | | |
|---|---|---|---|---|---|---|---|---|
| | Samples | | | | | | | |
| | | Early | 95% CI | | | | | |
| Gene | HV | OC | AUC | Lower | Upper | Sensitivity | Specificity | Accuracy |
| MMP10 | 20 | 6 | 0.8167 | 0.6538 | 0.9795 | 1.0000 | 0.7500 | 0.8077 |
| CDSN | 29 | 13 | 0.7851 | 0.6467 | 0.9236 | 0.9231 | 0.6207 | 0.7143 |
| MMP1 | 25 | 11 | 0.7527 | 0.5874 | 0.9180 | 1.0000 | 0.4800 | 0.6389 |
| INHBA | 28 | 11 | 0.7370 | 0.5501 | 0.9239 | 0.5455 | 0.8929 | 0.7949 |
| AIM2 | 20 | 9 | 0.7222 | 0.5330 | 0.9114 | 1.0000 | 0.4500 | 0.6207 |
| MMP9 | 28 | 11 | 0.6786 | 0.5022 | 0.8549 | 0.8182 | 0.6071 | 0.6667 |
| MMP13 | 9 | 4 | 0.7500 | 0.4715 | 1.0000 | 1.0000 | 0.6667 | 0.7692 |
| CRISP3 | 14 | 6 | 0.7500 | 0.4530 | 1.0000 | 0.8333 | 0.7143 | 0.7500 |
| MUC21 | 11 | 6 | 0.7424 | 0.4748 | 1.0000 | 0.6667 | 0.8182 | 0.7647 |
| MMP3 | 9 | 6 | 0.7037 | 0.4091 | 0.9983 | 0.8333 | 0.6667 | 0.7333 |
| ADAM12 | 27 | 10 | 0.6259 | 0.4062 | 0.8456 | 0.3000 | 0.9630 | 0.7838 |
| ISG15 | 26 | 11 | 0.5979 | 0.3958 | 0.8000 | 0.9091 | 0.3846 | 0.5405 |
| KRT4 | 13 | 6 | 0.5641 | 0.1896 | 0.9386 | 0.5000 | 0.9231 | 0.7895 |
| PTHLH | 29 | 12 | 0.5287 | 0.3176 | 0.7399 | 0.6667 | 0.5172 | 0.5610 |
| COL10A1 | 29 | 12 | 0.5172 | 0.3157 | 0.7188 | 0.8333 | 0.2759 | 0.4390 |
| COL1A1 | 27 | 10 | 0.5148 | 0.3057 | 0.7240 | 0.8000 | 0.4444 | 0.5405 |
| LAMC2 | 26 | 11 | 0.5000 | 0.3132 | 0.6868 | 0.9091 | 0.3077 | 0.4865 |
| CA9 | 2 | 1 | NA | NA | NA | NA | NA | NA |
| COL5A1 | 1 | 1 | NA | NA | NA | NA | NA | NA |
| MMP11 | 1 | 0 | NA | NA | NA | NA | NA | NA |

ROC curves from the genes with significant AUCs are the top six genes listed in Table 6 and shown in FIG. 12. Notably, the five genes with statistically different means (t-test) (i.e., AIM2, CDSN, INHBA, MMP1, and MMP10) were also found to have statistically significant AUC by ROC analysis. ROC analysis identified one additional gene, MMP9, not identified by the t-test, albeit with a small but significant AUC (0.6786).

### Example 12—ROC Analysis—Genes Combined

Based on the performance of the individual genes, gene expression levels were combined by logistic regression and performance evaluated by ROC analysis. Results are shown in FIG. 13 and Table 7. Combined gene expression showed improved performance in classification with a larger AUC compared to the AUC for an individual gene, especially in the three, four and five gene combinations (Table 6). The associated performance metrics (sensitivity, specificity and accuracy) at the optimal cut-off are listed with each AUC.

TABLE 7

| | Number of | | | | | | | | |
|---|---|---|---|---|---|---|---|---|---|
| | Samples | | | | | | | | |
| | | Early | 95% CI | | | | | | Youden |
| Gene | HV | OC | AUC | Lower | Upper | Sensitivity | Specificity | Accuracy | Index |
| CDSN + AIM2 | 20 | 9 | 0.8611 | 0.7160 | 1.0000 | 0.7778 | 0.8500 | 0.8276 | 0.6278 |
| CDSN + INHBA | 28 | 11 | 0.8279 | 0.6907 | 0.9652 | 0.9091 | 0.6786 | 0.7436 | 0.5877 |
| CDSN + MMP1 | 24 | 11 | 0.7955 | 0.6469 | 0.9440 | 0.9091 | 0.6667 | 0.7429 | 0.5758 |
| CDSN + MMP9 | 28 | 11 | 0.7597 | 0.6108 | 0.9087 | 0.9091 | 0.6429 | 0.7179 | 0.5520 |
| CDSN + MMP10 | 20 | 6 | 0.7917 | 0.6036 | 0.9798 | 1.0000 | 0.6500 | 0.7308 | 0.6500 |
| CDSN + AIM2 + | 17 | 8 | 0.9412 | 0.8219 | 1.0000 | 0.8750 | 1.0000 | 0.9600 | 0.8750 |
| MMP1 | | | | | | | | | |
| CDSN + AIM2 + | 20 | 8 | 0.9125 | 0.8023 | 1.0000 | 0.8750 | 0.9000 | 0.8929 | 0.7750 |
| INHBA | | | | | | | | | |
| CDSN + INHBA + | 24 | 10 | 0.8625 | 0.7216 | 1.0000 | 0.9000 | 0.7500 | 0.7941 | 0.6500 |
| MMP1 | | | | | | | | | |
| CDSN + AIM2 + | 14 | 4 | 0.8571 | 0.6541 | 1.0000 | 1.0000 | 0.7143 | 0.7778 | 0.7143 |
| MMP10 | | | | | | | | | |
| CDSN + INHBA + | 27 | 10 | 0.8556 | 0.7141 | 0.9970 | 0.9000 | 0.7778 | 0.8108 | 0.6778 |
| MMP9 | | | | | | | | | |
| CDSN + AIM2 + | 20 | 8 | 0.8500 | 0.7063 | 0.9937 | 0.8750 | 0.8000 | 0.8214 | 0.6750 |
| MMP9 | | | | | | | | | |
| CDSN + INHBA + | 20 | 5 | 0.8500 | 0.6288 | 1.0000 | 0.8000 | 0.8000 | 0.8000 | 0.6000 |
| MMP10 | | | | | | | | | |
| CDSN + AIM2 + | 17 | 7 | 0.9496 | 0.8585 | 1.0000 | 0.8571 | 0.9412 | 0.9167 | 0.7983 |
| INHBA + MMP1 | | | | | | | | | |
| CDSN + AIM2 + | 20 | 7 | 0.9214 | 0.8060 | 1.0000 | 0.8571 | 0.9500 | 0.9259 | 0.8071 |
| INHBA + MMP9 | | | | | | | | | |
| CDSN + AIM2 + | 14 | 3 | 0.9524 | 0.8395 | 1.0000 | 1.0000 | 0.8571 | 0.8824 | 0.8571 |
| INHBA + MMP10 | | | | | | | | | |
| CDSN + AIM2 + | 17 | 7 | 0.9664 | 0.9039 | 1.0000 | 1.0000 | 0.8235 | 0.8750 | 0.8235 |
| INHBA + MMP1 + | | | | | | | | | |
| MMP9 | | | | | | | | | |

As seen in FIG. 13, ROC curves for the two, three, four and five gene combinations have the largest AUC from each combination, with each of these gene combinations having a larger AUC than any of the single genes. The gene combination with the best performance as determined from the Youden index was observed with the three gene combination CDSN+AlM2+MMI (Youden index=0.8750) which resulted in an AUC of 0.9412, with a 0.8750 sensitivity and 1.000 specificity.

That which is claimed is:

1. A method to measure the presence and/or amount of a biomarker associated with Head and Neck Squamous Cell Carcinoma (HNSCC) in an individual comprising the steps of:
(a) obtaining a saliva sample from the individual; and,
(b) measuring in the saliva sample an amount of an expression product from at least one gene encoding the biomarkers associated with HNSCC, wherein the at least one gene comprises at least one of AIM2, CDSN, INHBA, MMP1, MMP9 or MMP10.

2. The method of claim 1, further comprising measuring the expression product from at least one of MMP13, CRISP3, MUC21, ADAM12, MMP3 or ISG15.

3. The method of claim 1, further comprising measuring the expression product of a housekeeping gene and normalizing the results.

4. A method of identifying an individual at risk for Head and Neck Squamous Cell Carcinoma (HNSCC), comprising the steps of:
(a) obtaining a saliva sample from the individual; and,
(b) measuring in the saliva sample an amount of an expression product from at least one gene encoding the biomarkers associated with HNSCC, wherein the at least one gene comprises at least one of AIM2, CDSN, INHBA, MMP1, MMP9, or MMP10, wherein the presence of an altered level of the expression product as compared to a healthy control for the biomarker associated with HNSCC identifies the individual as being at risk for HNSCC,
or
measuring in the saliva sample an altered amount of an expression product from the at least one gene encoding the biomarkers associated with HNSCC.

5. The method of claim 4, further comprising measuring the expression product from at least one of MMP13, CRISP3, MUC21, ADAM12, MMP3 or ISG15.

6. The method of claim 4, further comprising measuring expression of a housekeeping gene and normalizing the results.

7. A method of identifying an individual with Head and Neck Squamous Cell Carcinoma (HNSCC) and treating the individual, comprising the steps of:
(1) performing the method of claim 4; and,
(c) administering to the individual one or more HNSCC treatments.

8. A method of identifying an individual at risk for Head and Neck Squamous Cell Carcinoma (HNSCC) and monitoring the individual, comprising the steps of:
(a) obtaining a saliva sample from the individual;
(b) measuring in the saliva sample an increased amount of an expression product from at least one gene encoding the biomarkers associated with HNSCC, wherein the at least one gene comprises at least one of AIM2, CDSN, INHBA, MMP1, MMP9, or MMP10;
(c) determining that the at least one of AIM2, CDSN, INHBA, MMP1, MMP9, or MMP10 have altered expression as compared to a healthy control; and
(d) repeating steps (a)-(c) at a later time-point to determine if the at least one of AIM2, CDSN, INHBA, MMP1, MMP9, or MMP10 shows an increase in altered expression as compared to a healthy control.

9. The method of claim 8, further comprising measuring the expression product from the at least one of MMP13, CRISP3, MUC21, ADAM12, MMP3 or ISG15 in step (b) and determining that the at least one of MMP13, CRISP3, MUC21, ADAM12, MMP3 or ISG15 have altered expression in step (c) and repeating steps (a)-(c) at a later time-point to determine if the at least one of MMP13, CRISP3, MUC21, ADAM12, MMP3 or ISG15 shows an increase in altered expression as compared to a healthy control in step (d).

10. The method of claim 8, further comprising measuring the expression product from at least one of any of the genes shown in Tables 1, 2, 5 or 6 in step (b), determining that at least one of any of the genes shown in Tables 1, 2, 5 or 6 have altered expression in step (c) and repeating steps (a)-(c) at a later time-point to determine if the at least one of any of the genes shown in Tables 1, 2, 5 or 6 shows an increase in altered expression as compared to a healthy control in step (d).

11. The method of claim 8, further comprising measuring expression of a housekeeping gene and normalizing the results.

12. The method of claim 1, wherein the expression product is a protein or an mRNA.

13. The method of claim 1, wherein the step of measuring comprises duplex ddPCR or multiplex ddPCR.

14. A composition for performing the method of claim 1, comprising a reagent for detection of at least one of AIM2, CDSN, INHBA, MMP1, MMP9, or MMP10.

15. A kit comprising the composition of claim 14, and instructions for use of the kit.

16. The kit of claim 15, further comprising a reagent to detect the expression product from at least one of MMP13, CRISP3, MUC21, ADAM12, MMP3 or ISG15.

17. The kit of claim 15, further comprising a reagent to detect the expression product from at least one of any of the genes shown in Tables 1, 2, 5 or 6.

18. The kit of claim 15, further comprising a reagent to detect the expression product of a housekeeping gene for normalizing the results.

19. A system for performing any or all of the steps of the method of claim 1.

20. A computer-program product tangibly embodied in a non-transitory machine-readable storage medium, including instructions configured to run the system of claim 19.
